(19) United States
(12) Patent Application Publication
(10) Pub. No.: US 2006/0078916 A1
(43) Pub. Date: Apr. 13, 2006

(54) HM74 and HM74a in cuboidal endothelial cells as associated with inflammation

(76) Inventors: Luc Aguilar, Escalquens (FR); Alexandra Iche, Corronsac (FR); Regis Gayon, Ramonville (FR)

Correspondence Address: KNOBBE MARTENS OLSON & BEAR LLP, 2040 MAIN STREET, FOURTEENTH FLOOR, IRVINE, CA, 92614, US

(21) Appl. No.: 11/212,110

(22) Filed: Aug. 25, 2005

Related U.S. Application Data
(60) Provisional application No. 60/605,028, filed on Aug. 27, 2004.

(51) Int. Cl. C12Q 1/68; G01N 33/567

(52) U.S. Cl. 435/6; 435/7.2

(57) ABSTRACT

Disclosed herein are methods of diagnosing and monitoring the severity of inflammatory diseases. Also disclosed herein are methods for screening for compounds which are effective at reducing the symptoms associated with an inflammatory disorder. Such compounds can be used in the methods of treating inflammatory disorders as disclosed herein.

### RELATED APPLICATIONS

The application is a nonprovisional application of and claims priority to U.S. Provisional Patent Application No. 60/605,028, entitled HM74 AND HM74A IN CUBOIDAL ENTOTHELIAL CELLS AS ASSOCIATED WITH INFLAMMATION, filed Aug. 27, 2004.

### FIELD OF THE INVENTION

The present invention relates to the fields of medicine and cell biology. In particular, the present invention relates to methods of using the cuboidal endothelial cell-associated G-protein coupled receptors in therapeutic, diagnostic and screening applications.

### BACKGROUND

Rheumatoid arthritis (RA) is a chronic, systemic inflammatory disease characterized by pain and destruction of the joints. The synovium or synovial membrane, which surround the joint cavity, becomes massively hypertrophied in individuals having RA. Synovial tissue, also known as pannus, can become so invasive that it penetrates and degrades the cartilage and bone, thus potentially resulting in joint deformities, functional deterioration and profound disabilities.

Rheumatoid arthritis affects between 0.5 and 1% of the population. This disease affects both males and females of all ethinic groups. RA can occur at any age, but the peak incidence of the disease occurs between the age of 40 and 60. The impact of RA is enormous both at an individual level and from a wider socioeconomic perspective.

Crohn's disease (CD) is an inflammatory bowel disease, which causes inflammation in the small intestine. About 500,000 Americans are afflicted with Crohn's disease. The disease affects men and women equally and seems to run in some families. Although fewer people suffer from CD than RA, the effects of CD can greatly impact an individual's daily life.

In spite of considerable research into therapies for these disorders, inflammatory diseases, such as rheumatoid arthritis and Crohn's diesease, remain difficult to effectively diagnose and treat. For example, CD is difficult to diagnosis because it often mimics other gastrointestinal disorders and because the symptoms can vary widely. Similarly, there are no reliable diagnostic tests for RA, nor are there suitable tests that would permit monitoring of the course of this disease in patients. With RA, these problems are exacerbated by the fact that this disease frequently goes undiagnosed because patients often have no pain. Although many patients experience no pain, RA may be actively destroying the patient's joint tissue. Other patients, who are known to have symptoms, can be treated to alleviate such symptoms, but the rate of progression of joint destruction cannot easily be monitored. As such, there exists a need for methods of diagnosing, monitoring the progression of inflammatory diseases such as RA and CD.

Drug therapy is available for some inflammatory diseases, but the most effective medicines are toxic (for example, steroids and methotrexate), and thus, they need to be used with caution. TNF blockers are very effective against RA, but not all patients respond to these drugs. Furthermore, these drugs are expensive and have some serious side effects, which include immune suppression, toxicity to organ systems, allergy and metabolic disturbances. Although several groups of drugs are available for treating CD, none are highly effective and most have undesirable side effects.

Data from longitudinal clinical and epidemiologic studies provide guidelines for treatments. These studies emphasize: 1) the need for early diagnosis, 2) identification of prognostic factors, and 3) early aggressive treatment. For example, improvement in the diagnosis, monitoring and treatment of RA may help prevent irreversible joint damage. As such, improved methods and reagents for the diagnosis, characterization, prognosis, monitoring and treatment of inflammatory diseases, such as RA and CD, are needed.

### SUMMARY OF THE INVENTION

Some embodiments of the present invention, are directed to methods of determining and/or diagnosing whether a subject is afflicted with an inflammatory disorder. Exemplary inflammatory disorders include, but are not limited to, joint disorders, such as rheumatoid arthritis (RA), and gastrointestinal disorders, such as Crohn's disease (CD). These methods typically include the step of obtaining a biological sample from the subject to be tested. Common biological samples include, but are not limited to, soft tissue samples, hard tissue samples and bodily fluid samples, such as blood and lymph. The biological sample is then analyzed to determine the level of expression of one or more markers in these samples, particularly in cuboidal endothelial cells.

In related embodiments of the present invention, a biological sample, which is obtained from a subject, is tested to determine whether the sample comprises cells displaying a pattern or profile of expression of a marker set which correlates with one or more inflammatory disorders.

Other aspects of the present invention relate to newly discovered functions of HM74 and HM74a as cuboidal endothelial cell markers that are associated with inflammatory diseases, such as RA. In particular embodiments, it has been discovered that an increased level of expression of these markers in cuboidal endothelial cells of inflamed tissues, as compared with the expression of these markers in non-inflamed tissues, correlates with RA. As such, the methods described herein relate to detecting the presence or absence of RA, determining the type and severity of RA, and screening compounds to evaluate whether they are effective in reducing the symptoms associated with RA.

In some embodiments of the present invention, the level of expression of an HM74 or HM74a gene product in a sample can be assessed by any method known in the art for detecting the presence and/or the amount of a gene product in a sample. For example, the presence or amount of an HM74 or HM74a gene product in a sample can be determined by detecting the presence of a marker protein having a polypeptide sequence selected from the group consisting of SEQ ID NOs: 3, 6, or 14, polypeptide sequences substantially homologous to any of SEQ ID NOs: 3, 6, or 14, and fragments any of these sequences. In such embodiments, the protein can be detected by using a detector reagent, such as an antibody, an antibody derivative, or an antibody fragment, which binds specifically with the marker protein or a fragment of the protein. In other embodiments, a metabolite which is produced directly or indirectly by a marker protein is detected. In still other embodiments, the presence or amount of a transcribed polynucleotide encoding HM74 or HM74a in a sample can be determined by detecting the presence of a marker polynucleotide a having a sequence selected from the group consisting of SEQ ID NOs: SEQ ID NOs: 1, 2, 4, 5, or 7-13, polynucleotide sequences substantially homologous to any of SEQ ID NOs: 1, 2, 4, 5, or 7-13, and fragments any of these polynucleotide sequences.

Other aspects of the invention are described in the following numbered paragraphs:

1. A method of detecting the expression of a gene product, said method comprising:
obtaining a cuboidal endothelial cell; and
determining whether one or more gene products selected from the group consisting of HM74, HM74a and homologs thereof is expressed in said cuboidal endothelial cell.

2. The method of Paragraph 1, wherein the expression of one or more gene products selected from the group consisting of HM74, HM74a and homologs thereof is determined by quantitative polymerase chain reaction (Q-PCR).

3. The method of Paragraph 1, wherein the expression of one or more gene products selected from the group consisting of HM74, HM74a and homologs thereof is determined by a binding a detector reagent thereto.

4. The method of Paragraph 3, wherein the said detector reagent is an antibody.

5. The method of Paragraph 1, wherein said one or more gene products is HM74.

6. The method of Paragraph 1, wherein said one or more gene products is HM74a.

7. A method of monitoring the severity of an inflammatory disorder, said method comprising the steps of:
obtaining a biological sample of an inflamed tissue, said biological sample comprising cuboidal endothelial cells;
measuring the expression level of one or more gene products in said cuboidal endothelial cells, wherein said one or more gene products is selected from the group consisting of HM74, HM74a and homologs thereof; and
determining whether the expression level of said one or more gene products selected from the group consisting of HM74, HM74a and homologs thereof in said cuboidal endothelial cells is increased relative to the expression level of said one or more gene products selected from the group consisting of HM74, HM74a and homologs thereof in endothelial cells present in a non-inflamed tissue, wherein an increased expression level of said one or more gene products selected from the group consisting of HM74, HM74a and homologs thereof in said cuboidal endothelial cells indicates increased severity of said inflammatory disorder.

8. The method of Paragraph 7 further comprising isolating endothelial cells from said biological sample.

9. The method of Paragraph 7, wherein said inflammatory disorder is rheumatoid arthritis.

10. The method of Paragraph 7, wherein said biological sample is synovial tissue.

11. The method of Paragraph 7, wherein said expression level of said one or more gene products selected from the group consisting of HM74, HM74a and homologs thereof is measured by Q-PCR.

12. The method of Paragraph 7, wherein said expression level of said one or more gene products selected from the group consisting of HM74, HM74a and homologs thereof is measured by immunohistochemistry.

13. The method of Paragraph 7, wherein said determining step comprises determining whether said expression level of said one or more gene products selected from the group consisting of HM74, HM74a and homologs thereof in cuboidal endothelial cells is at least about 2-fold greater than the expression of said one or more gene products selected from the group consisting of HM74, HM74a and homologs thereof in endothelial cells from non-inflamed tissue.

14. The method of Paragraph 7, wherein said determining step comprises determining whether said expression level of said one or more gene products selected from the group consisting of HM74, HM74a and homologs thereof in cuboidal endothelial cells is at least about 10-fold greater than the expression of said one or more gene products selected from the group consisting of HM74, HM74a and homologs thereof in endothelial cells from non-inflamed tissue.

15. The method of Paragraph 7, wherein said determining step comprises determining whether said expression level of said one or more gene products selected from the group consisting of HM74, HM74a and homologs thereof in cuboidal endothelial cells is at least about 20-fold greater than the expression of said one or more gene products selected from the group consisting of HM74, HM74a and homologs thereof in endothelial cells from non-inflamed tissue.

16. The method of Paragraph 7, wherein said determining step comprises determining whether said expression level of said one or more gene products selected from the group consisting of HM74, HM74a and homologs thereof in cuboidal endothelial cells is at least about 50-fold greater than the expression of said one or more gene products selected from the group consisting of HM74, HM74a and homologs thereof in endothelial cells from non-inflamed tissue.

17. The method of Paragraph 7, wherein said determining step comprises determining whether said expression level of said one or more gene products selected from the group consisting of HM74, HM74a and homologs thereof in cuboidal endothelial cells is at least about 100-fold greater than the expression of said one or more gene products selected from the group consisting of HM74, HM74a and homologs thereof in endothelial cells from non-inflamed tissue.

18. The method of Paragraph 7, wherein said one or more gene products is HM74.

19. The method of Paragraph 7, wherein said one or more gene products is HM74a.

20. A method of diagnosing an inflammatory disorder, said method comprising:
obtaining a biological sample of an inflamed tissue, said biological sample comprising cuboidal endothelial cells;
determining whether one or more gene products selected from the group consisting of HM74, HM74a and homologs thereof is expressed in said cuboidal endothelial cells, wherein the presence of said one or more gene products selected from the group consisting of HM74, HM74a and homologs thereof in said cuboidal endothelial cells is indicative of an inflammatory disorder.

21. The method of Paragraph 20, wherein said inflammatory disorder is rheumatoid arthritis.

22. The method of Paragraph 20, wherein said biological sample is synovial tissue.

23. The method of Paragraph 20, wherein Q-PCR is used to determine whether one or more gene products selected from the group consisting of HM74, HM74a and homologs thereof is expressed in said cuboidal endothelail cells.

24. The method of Paragraph 20, wherein immunohistochemistry is used to determine whether one or more gene products selected from the group consisting of HM74, HM74a and homologs thereof is expressed in said cuboidal endothelail cells.

25. The method of Paragraph 20, wherein said one or more gene products is HM74.

26. The method of Paragraph 20, wherein said one or more gene products is HM74a.

27. A method of evaluating whether a test compound is effective at reducing the symptoms of an inflammatory disorder, said method comprising the steps of:
determining the expression level of one or more gene products selected from the group consisting of HM74, HM74a and homologs thereof in a cuboidal endothelial cell;
contacting said cuboidal endothelial cell with a test compound;
determining the expression level of said one or more gene products selected from the group consisting of HM74, HM74a and homologs thereof in said cuboidal endothelial cell that has been contacted with said test compound;
determining whether the expression level of said one or more gene products selected from the group consisting of HM74, HM74a and homologs thereof in said cuboidal endothelial cell that has been contacted with said test compound is increased relative to the expression level of said one or more gene products selected from the group consisting of HM74, HM74a and homologs thereof in said cuboidal endothelial cell that has not been contacted with said test compound, wherein a reduced expression level of said one or more gene products selected from the group consisting of HM74, HM74a and homologs thereof in said cuboidal endothelial cell that has been contacted with said test compound relative to the expression level of said one or more gene products selected from the group consisting of HM74, HM74a and homologs thereof in said cuboidal endothelial cell that has not been contacted with said test compound indicates that said test compound is effective at reducing the symptoms of an inflammatory disorder.

28. The method of Paragraph 27, wherein said inflammatory disorder is rheumatoid arthritis.

29. The method of Paragraph 27, wherein said expression level of said one or more gene products selected from the group consisting of HM74, HM74a and homologs thereof is measured by Q-PCR.

30. The method of Paragraph 27, wherein said expression level of said one or more gene products selected from the group consisting of HM74, HM74a and homologs thereof is measured by immunohistochemistry.

31. The method of Paragraph 27, wherein said one or more gene products is HM74.

32. The method of Paragraph 27, wherein said one or more gene products is HM74a.

33. A method of evaluating whether a test compound is effective at reducing the symptoms of an inflammatory disorder, said method comprising determining whether said test compound reduces the expression level of one or more gene products selected from the group consisting of HM74 and HM74a in cuboidal endothelial cells.

34. The method of Paragraph 33, wherein said inflammatory disorder is rheumatoid arthritis.

35. The method of Paragraph 33, wherein said expression level of said one or more gene products selected from the group consisting of HM74, HM74a and homologs thereof is measured by Q-PCR.

36. The method of Paragraph 33, wherein said expression level of said one or more gene products selected from the group consisting of HM74, HM74a and homologs thereof is measured by immunohistochemistry.

37. The method of Paragraph 33, wherein said one or more gene products is HM74.

38. The method of Paragraph 33, wherein said one or more gene products is HM74a.

39. A method of evaluating whether a test compound is effective at reducing the symptoms of an inflammatory disorder, said method comprising:
determining whether said test compound binds to one or more gene products selected from the group consisting of HM74, HM74a and homologs thereof; and
evaluating a test compound which binds to one or more gene products selected from the group consisting of HM74, HM74a and homologs thereof in an animal model of said inflammatory disorder.

40. The method of Paragraph 39 further comprising determining whether said test compound reduces the expression level of said one or more gene products selected from the group consisting of HM74, HM74a and homologs thereof in cuboidal endothelial cells in said animal model.

41. The method of Paragraph 39, wherein said animal model is a mouse model.

42. The method of Paragraph 39, wherein said inflammatory disorder is rheumatoid arthritis.

43. The method of Paragraph 39, wherein said test compound is a small molecule.

44. The method of Paragraph 39, wherein said test compound is a natural product.

45. The method of Paragraph 39, wherein said test compound is a macromolecule.

46. The method of Paragraph 45, wherien said macromolecule is a polypeptide.

47. The method of Paragraph 46, wherein said polypeptide is an antibody.

48. A method of ameliorating the symptoms associated with an inflammatory disorder, said method comprising inhibiting the expression of one or more gene products selected from the group consisting of HM74, HM74a and homologs thereof in cuboidal endothelial cells of an inflamed tissue, thereby ameliorating the symptoms associated with said inflammatory disorder.

49. The method of Paragraph 48, wherein said inflammatory disorder is rheumatoid arthritis.

50. The method of Paragraph 48, wherein said tissue is synovial tissue.

51. The method of Paragraph 48, wherein said one or more gene products is HM74.

52. The method of Paragraph 48, wherein said one or more gene products is HM74a.

53. A method of inhibiting the progression of tissue damage associated with an inflammatory disorder, said method comprising reducing the level of expression of one or more genes selected from the group consisting of HM74, HM74a and homologs thereof in cuboidal endothelial cells of an inflamed tissue, thereby inhibiting the progression of tissue damage associated with said inflammatory disorder.

54. The method of Paragraph 53, wherein said inflammatory disorder is rheumatoid arthritis.

55. The method of Paragraph 53, wherein said tissue is synovial tissue.

56. The method of Paragraph 53, wherein said one or more genes is HM74.

57. The method of Paragraph 53, wherein said one or more genes is HM74a.

58. Use of an inhibitor of the expression of one or more gene products selected from the group consisting of HM74, HM74a and homologs thereof in cuboidal endothelial cells of an inflamed tissue for the preparation of a medicament for ameliorating the symptoms associated with an inflammatory disorder.

59. The use according to Paragraph 58, wherein said inflammatory disorder is rheumatoid arthritis.

60. The use according to Paragraph 58, wherein said tissue is synovial tissue.

61. The use according to Paragraph 58, wherein said one or more gene products is HM74.

62. The use according to Paragraph 58, wherein said one or more gene products is HM74a.

63. Use of an inhibitor of the expression of one or more genes selected from the group consisting of HM74, HM74a and homologs thereof in cuboidal endothelial cells of an inflamed tissue for the preparation of a medicament for inhibiting the progression of tissue damage associated with said inflammatory disorder.

64. The use according to Paragraph 63, wherein said inflammatory disorder is rheumatoid arthritis.

65. The use according to Paragraph 63, wherein said tissue is synovial tissue.

66. The use according to Paragraph 63, wherein said one or more genes is HM74.

67. The use according to Paragraph 63, wherein said one or more genes is HM74a.

It will be appreciated that any of the embodiments described above or elsewhere herein can be performed in vitro, in vivo or ex vivo. In some embodiments, one or more tissues comprising cuboidal endothelial cells can be removed from a subject or patient and then subjected to one or more of the methods described herein. Such embodiments constitute in vitro aspects of the present invention. In other embodiments, after subjecting one or more tissues comprising cuboidal endothelial cells, which have been removed from a subject or patient, to one or more of the methods described herein, the cuboidal endothelial cells are replaced. Such embodiments constitute ex vivo aspects of the present invention. In still other embodiments, one or more tissues comprising cuboidal endothelial cells can be located within a subject or patient, and without removing these one or more tissues, one or more of the methods described herein are performed.

### BRIEF DESCRIPTION OF THE DRAWINGS

FIGS. 1A-D show the results of an in situ hybridization experiment with HM74 riboprobes on inflammed RA sections. A strong HM74 antisense riboprobe signal is detected (B) and co-localized on cuboidal endothelial venules labelled by IHC with anti-DARC antibody on the same tissue section (A). The negative control, HM74 sense riboprobe, does not show any significant signal (D) on cuboidal endothelial cells (C). Original magnification, X200.

FIGS. 2A-D show the result of an in situ hybridization experiment with HM74 riboprobes on inflammed human tonsil sections. A strong HM74 antisense riboprobe signal is detected (B) and co-localized on cuboidal endothelial venules labelled by IHC with anti-DARC antibody on the same tissue section (A). The negative control, HM74 sense riboprobe, does not show any significant signal (D) on cuboidal endothelial cells (C). Original magnification, X400.

FIGS. 3A-D show the results of an in situ hybridization experiment with HM74 riboprobes on inflammed Crohn's disease sections. A strong HM74 antisense riboprobe signal is detected (B) and co-localized on cuboidal endothelial venules labelled by IHC with anti-DARC antibody on the same tissue section (A). The negative control, HM74 sense riboprobe, does not show any significant signal (D) on cuboidal endothelial cells (C). Original magnification, X100.

FIGS. 4A-D show the results of an in situ hybridization experiment with antisense HM74 riboprobes on non-inflammed sections. HM74 antisense riboprobe signal is not detected (B and D) on the few cuboidal endothelial venules labelled by IHC with anti-DARC antibody on the same tissue sections (A and C). FIGS. 4A and 4B: non-inflammed osteoarthritis sections. FIGS. 4C and 4D: non-inflammed CD sections. Original magnification, X200.

FIG. 5 shows the validation of differential expression of HM74 and HM74a in various cuboidal endothelial cells isolated from inflamed and non-inflammed samples using quantitative PCR. Differential relative mRNA expression is calculated using the 2(-Delta Delta C(T)) method with GAPDH as the reference and compared to the relative expression of HM74 and HM74a in endothelial cells from the non-inflammed OA sample.

FIG. 6 is a graph comparing the fold difference between the expression of HM74 in 6 inflammed RA samples and 5 non-inflammed OA samples. Fold difference is calculated as described in FIG. 5.

### DETAILED DESCRIPTION OF THE INVENTION

During the development of rheumatoid arthritis (RA), one of the earliest pathophysiological events in the synovium is the activation of the venular endothelium resulting in the migration of leukocytes from the blood across these cells into the tissue. In addition, endothelial cells proliferate, thus resulting in new blood vessel formation. During the first month of synovitis (inflammation of the synovium), changes to the venular endothelium include hypertrophy, characterized by the cells becoming cuboidal in morphology, the development of gaps between endothelial cells, and the presence of multiple concentric layers in the basement membrane. Associated with these changes is the transendothelial migration of numerous mononuclear and polymorphonuclear inflammatory cells.

The cuboidal morphology of the endothelial cells of synovial postcapillary venules resembles that of the endothelial cells of the high endothelial venules (“HEV”), which are the postcapillary venules of lymphoid tissues. (Girard and Springer (1995), Immunity 12:113-123; Girard et al. (1999), Am. J. Path. 155:2045-2055, the disclosures of which are incorporated herein by reference in their entireties). The postcapillary venules of the rheumatoid synovium in patients with active, uncontrolled disease exhibit HEV-like morphology, especially in the regions near lymphocyte aggregates, whereas tissues of patients whose disease has been modified by treatment exhibit a flatter endothelium.

Transendothelial migration of T cells across cuboidal endothelium is considerably shorter and more efficient than in flat endothelium. This unique feature of lymphocyte migration through cuboidal cell (HEVECs) layers probably results from a serie of highly specific interactions of molecules acting in a multistep process. (Springer, 1994). As such, determining the difference in gene expression between cuboidal cells from inflamed tissues and endothelial cells from non-inflamed tissues can lead to the identification of genes and gene products whose expression is associated with the inflammatory process. Genes and gene products that are associated with inflammation can lead to the identification of molecular markers of inflammatory disorders as well as targets with a high potential therapeutic value for the diagnosis, characterization, prognosis, monitoring and treatment of inflammatory disorders, such as RA.

As used herein, “gene product” refers to any product produced by a gene. Such products include, but are not limited to, pre-spliced hnRNA, mRNA, polysomes, preproproteins, proproteins, immature proteins, unmodified proteins and modified proteins, such as glycosylated, farnesylated, or phosphorylated proteins.

Recently, several methods and technologies have been developed for the detection of differential gene expression and the isolation of differentially expressed genes. One method, which can be used to identify differentially expressed genes is a subtractive suppressive hybridization (SSH, Clontech). SSH is a PCR-based subtractive enrichment procedure that enables isolation of genes with altered expression between various tissues or cell samples. This technique offers several advantages, including the isolation of few false positives, competitive elimination of unwanted difference products and detection and isolation of genes producing rare transcripts.

As mentioned above, the identification of genes, which have increased expression levels in cuboidal endothelial cells from inflammed tissues, provides an important link to diagnosing, monitoring and treating inflammatory disorders. Tissue distribution and disease association of the increased expression of these genes and gene products can be established using tissue samples derived from appropriately selected patients. Various techniques, for example, histology methods, such as in situ hybridization and immunohistochemistry, can be applied for this purpose.

Signals that are needed for leukocyte migration and activation are often communicated through receptors that belong to the seven transmembrane-spanning G-protein coupled receptor (GPCR) superfamily. The receptors of this superfamily are characterized by seven transmembrane helices (TM-I through TM-VII), which are connected by three intracellular and three extracellular loops. The GPCR gene family is the largest known receptor family. GPCRs are transducers of extracellular signals and they allow tissues to respond to a wide array of signalling molecules. G-protein coupled receptors are important targets in therapeutic applications because they are involved in a wide variety of physiological and pathological processes. It is estimated that 60-70% of currently marketed drugs indeed act on members of the GPCR superfamily.

In some embodiments of the present invention, the gene products having increased levels of expression in cuboidal endothelial cells of inflammed tissue are GPCRs. Identification of GPCR genes and gene products that are expressed in inflammatory cells and establishing the association of their increased expression with disease conditions provides an important opportunity for the understanding of the inflammatory conditions from which a number of clinically important applications arise. Furthermore, such identification of GPCR genes and gene products leads to the identification and the development of therapeutic compounds for ameliorating the symptoms associated with an inflammatory disease, such as RA. For example, therapeutic compounds that can be developed include, but are not limited to, small molecule drugs, antisense molecules and antibody molecules. Alternatively, a therapeutic compound can target a biochemical pathway at an upstream or downstream location in the GPCR pathway. Additionally, the GPCR genes and gene products that are identified can be used as diagnostic markers for or markers for monitoring inflammatory disorders.

HM74 and HM74a

Human HM74 was first cloned in 1993 by Nomura et al. (Int. Immunol. 5:1239-49). The HM74 polypeptide (SEQ ID NO: 3) is 387 amino acids in length and is encoded by a gene which has a coding strand sequence (SEQ ID NO: 2) that is 1164 nucleotides in length. Numerous variants, many of which are single nucleotide polymorphism (SNPs), in both the HM74 coding and noncoding sequence have been identified (SEQ ID NOs: 4, 5, and 7-13).

HM74 is a transmembrane-spanning GPCR that appears to be expressed in mononuclear cells and neutrophils. HM74 has been demonstrated to possesses a low affinity for nicotinic acid; however, it is unlikely that this molecule is the natural ligand for this GPCR since the physiological concentration of nicotinic acid that is available in the plasma is much lower than the concentration needed to activate this receptor.

A second related GPCR, HM74a, was identified as a high affinity GPCR for nicotinic acid in 2003 by Soga, et al. (Biochem. Biophys. Res. Commun. 303:364-369). The HM74a polypeptide (SEQ ID NO: 6) is 363 amino acids in length and displays about 95% amino acid sequence identity with its paralog, HM74. The coding strand sequence (SEQ ID NO: 5) comprises 1092 nucleotides.

Variant nucleotide sequences for both HM74 and HM74a can be obtained from the National Center for Biotechnology Information (NCBI). A website for NCBI, which provides access to, among other things, searchable nucleic acid and protein sequence databases, can be found by entering into the address bar of a web browser the text, “www.ncbi.nlm” immediately followed by “nih.gov”. The disclosure of the amino acid sequences for both HM74 and HM74a (having Genbank Accession Nos. BAA01721 and NP_{—}808219, respectively) are incorporated herein by reference in their entireties. Additionally, the nucleotide sequences for both HM74 and HM74a (having Genbank Accession Nos. D10923 and NM_{—}177551, respectively) as well as nucleotide sequence variants of these sequences are incorporated herein by reference in their entireties.

Methods for Obtaining Variant Nucleic Acids and Polypeptides

Nucleic acids and polypeptides sharing sequence homology with HM74 and HM74a sequences can be identified by methods known in the art, which include, but are not limited to, nucleic acid hybridization and database searching.

With reference to nucleic acids, “homologous sequence” means a nulceotide sequence having at least 70%, at least 72%, at least 74%, at least 76%, at least 78%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99% or at least 99.5% nucleotide sequence identity with a nucleotide sequence selected from the group consisting of SEQ ID NOs. 1, 2, 4, 5, and 7-13.

With reference to polypeptides, “homologous sequence” means an amino acid sequence having at least 50%, at least 55%, at least 60%, at least 65%, at least 70%, at least 72%, at least 74%, at least 76%, at least 78%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99% or at least 99.5% amino acid sequence identity with an amino acid sequence selected from the group consisting of SEQ ID NOs. 3, 6, and 14.

Homologous nucleic acids and polypeptides can be identified by database searching using algorithms such as BLAST and FASTA. For example, the identity or similarity between HM74 or HM74a and sequence homologs may be determined using the FASTA version 3.0t78 algorithm with the default parameters. Alternatively, protein identity or similarity may be determined using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Homologous nucleic acids can be identified by nucleic acid hybridization under stringent or moderate conditions. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, for example, to washing in 6×SSC/0.05% sodium pyrophosphate at 37° C., 48° C., 55° C., and 60° C. as appropriate for the particular probe being used. As used herein, “moderate conditions” means hybridization to filter-bound DNA in 6× sodium chloride/sodium citrate (SSC) at about 45° C. followed by one or more washes in 0.2×SSC/0.1% SDS at about 42-65° C.

Nucleic acid hybridization may be under stringent or moderate conditions as defined above or under other conditions which permit specific hybridization. The nucleic acid molecules that hybridize to the nucleic acid coding sequences include oligodeoxynucleotides which hybridize to the target nucleic under highly stringent or stringent conditions. In general, for oligonucleotides between 14 and 70 nucleotides in length the melting temperature (Tm) is calculated using the formula:
Tm(° C.)=81.5+16.6(log [monovalent cations (molar)]+0.41 (% G+C)−(500/N)

where N is the length of the probe. If the hybridization is carried out in a solution containing formamide, the melting temperature may be calculated using the equation:
Tm(° C.)=81.5+16.6(log [monovalent cations (molar)]+0.41(% G+C)−(0.61)(% formamide)−(500/N)

where N is the length of the probe. In general, hybridization is carried out at about 20-25 degrees below Tm (for DNA-DNA hybrids) or about 10-15 degrees below Tm (for RNA-DNA hybrids).

Other hybridization conditions are apparent to those of skill in the art (see, for example, Ausubel, F. M. et al., eds., 1989, Current Protocols in Molecular Biology, Vol. I, Green Publishing Associates, Inc. and John Wiley & Sons, Inc., New York, at pp. 6.3.1-6.3.6 and 2.10.3, the disclosure of which is incorporated herein by reference in its entirety).

In addition to naturally-occurring allelic variants of the HM74 and HM74a sequences, a skilled artisan will appreciate that changes can be introduced by mutation into the nucleotide sequences of SEQ ID NOs: 1, 2, 4, 5, or 7-13, thereby leading to changes in the amino acid sequence of the encoded HM74 and HM74a, with or without altering the function or expression level of these gene products.

Several types of variants are contemplated including 1) one in which one or more of the amino acid residues are substituted with a conserved or non-conserved amino acid residue and such substituted amino acid residue may or may not be one encoded by the genetic code, or 2) one in which one or more of the amino acid residues includes a substituent group, or 3) one in which the mutated HM74 or HM74a is fused with another compound, or 4) one in which the additional amino acids are fused to HM74 or HM74a, such as a leader or secretory sequence or a sequence which is employed for purification of the HM74 or HM74a polypeptide. Such variants are deemed to be within the scope of those skilled in the art.

For example, nucleotide substitutions leading to amino acid substitutions can be made in the sequences of SEQ ID NOs: 1, 2, 4, 5, or 7-13 that do not substantially change the biological activity of the protein. An amino acid residue can be altered from the wild-type sequence encoding a an HM74 or HM74a polypeptide without altering the biological activity. In general, amino acid residues that are conserved between HM74, HM74a and other GPCRs are predicted to be less amenable to alteration.

In one aspect, the invention pertains to nucleic acid molecules encoding HM74 or HM74a polypeptides, or biologically active fragments or homologs thereof that contain changes in amino acid residues that are not essential for activity or nucleic acids encoding fragments or homologs thereof where the nucleic acid is capable of being used as a probe to determine the level of a nucleic acid encoding HM74 or HM74a or a variant or homolog thereof in a sample. Such proteins differ in amino acid sequence from SEQ ID NOs: 3, 6, or 14 yet retain biological activity. In one embodiment, the HM74 or HM74 a nucleic acid molecule comprises a nucleotide sequence encoding a protein, wherein the protein comprises an amino acid sequence at least about 50-60% homologous to an amino acid sequence selected from the group consisting of SEQ ID NOs: 3, 6, and 14. Preferably, the protein encoded by the nucleic acid molecule is at least about 65-70% homologous to an amino acid sequence selected from the group consisting of SEQ ID NOs: 3, 6, and 14, more preferably sharing at least about 75-80% identity with an amino acid sequence selected from the group consisting of SEQ ID NOs: 3, 6, and 14, even more preferably sharing at least about 85%, 90%, 92%, 95%, 97%, 98%, 99% or 99.8% identity with an amino acid sequence selected from the group consisting of SEQ ID NOs: 3, 6, and 14.

An isolated nucleic acid molecule encoding an HM74 or HM74a polypeptide homologous to a protein of any one of SEQ ID NOs: 3, 6, or 14 can be created by introducing one or more nucleotide substitutions, additions or deletions into the nucleotide sequence of SEQ ID NOs: 1, 2, 4, 5, or 7-13 such that one or more amino acid substitutions, additions or deletions are introduced into the encoded protein. Mutations can be introduced into any of SEQ ID NOs: 1, 2, 4, 5, or 7-13, by standard techniques, such as site-directed mutagenesis and PCR-mediated mutagenesis. For example, conservative amino acid substitutions may be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an HM74 or HM74a polypeptide can be replaced with another amino acid residue from the same side chain family.

The invention also provides HM74 or HM74a chimeric or fusion proteins. As used herein, an HM74 or HM74a “chimeric protein” or “fusion protein” comprises an HM74 or HM74a polypeptide or portion thereof operatively linked, preferably fused in frame, to a non-HM74 or HM74a polypeptide. In some embodiments, such fusion proteins can facilitate the purification of recombinant HM74 and HM74a polypeptides or portions thereof.

Expression of HM74 and/or HM74a in a Host Cells

Another aspect of the present invention pertains to vectors, preferably expression vectors, containing a nucleic acid encoding an HM74 or HM74a polypeptide, or a biologically active fragment or homolog thereof or vectors comprising a nucleic acid which can be used to measure the level of a nucleic acid encoding HM74 or HM74a or a homolog thereoof in a sample. In another embodiment, the vectors encode a portion of HM74 or HM74a or a homolog hereof which can be used to generate antibodies which recognize HM74 or HM74a or a homolog thereof.

Vectors may have particular use in the preparation of a recombinant protein of the invention. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In some embodiments the recombinant expression vectors of the invention comprise an HM74 or HM74a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (for example, in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990), the disclosure of which is incorporated herein by reference in its entirety. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, as well as other factors. The expression vectors of the invention can be introduced into host cells to thereby produce proteins or peptides, including fusion proteins or peptides, encoded by nucleic acids as described herein (for example, HM74 and HM74a proteins, mutant forms of HM74 and HM74a proteins, HM74 and HM74a fusion proteins, homologs of HM74 and HM74a proteins or fragments of any of the preceding proteins).

The recombinant expression vectors of the invention can be designed for expression of an HM74 or HM74a polypeptide, or a biologically active fragment or homolog thereof in prokaryotic or eukaryotic cells. For example, HM74 or HM74a proteins can be expressed in bacterial cells such as E. coli, insect cells (using baculovirus expression vectors) yeast cells, or mammalian cells. Suitable host cells are discussed further in Goeddel, Gene Expression Technology: Methods in Enzymology 185, Academic Press, San Diego, Calif. (1990), the disclosure of which is incorporated herein by reference in its entirety. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.), the disclosures of which are incorporated herein by reference in their entireties, which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89), the disclosures of which are incorporated herein by reference in their entireties. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn 1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128, the disclosure of which is incorporated herein by reference in its entirety). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118, the disclosure of which is incorporated herein by reference in its entirety). Such alteration of nucleic acid sequences of the invention can be carried out by standard DNA synthesis techniques.

In another embodiment, the HM74 or HM74a expression vector is a yeast expression vector. Examples of vectors for expression in yeast S. cerivisae include pYepSec 1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.), the disclosures of which are incorporated herein by reference in their entireties.

Alternatively, HM74 or HM74a proteins or fragments or homologs thereof can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39), the disclosures of which are incorporated herein by reference in their entireties. In particularly preferred embodiments, HM74 or HM74a proteins are expressed according to Karniski et al, Am. J. Physiol. (1998) 275: F79-87, the disclosure of which is incorporated herein by reference in its entirety.

In yet another embodiment, an HM74 or HM74a polypeptide or a fragment or homolog thereof is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195), the disclosures of which are incorporated herein by reference in their entireties. When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989, the disclosure of which is incorporated herein by reference in its entirety. In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art, and are further described below.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to HM74 or HM74a mRNA, an mRNA encoding a homolog of HM74 or HM74a, or a portion of any of the preceding mRNAs. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986, the disclosure of which is incorporated herein by reference in its entirety.

Another aspect of the invention pertains to host cells into which a recombinant expression vector of the invention has been introduced. The terms “host cell” and “recombinant host cell” are used interchangeably herein. It is understood that such term refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an HM74 or HM74a protein can be expressed in bacterial cells such as E. coli, insect cells, yeast or mammalian cells (such as Chinese hamster ovary cells (CHO) or COS cells or human cells). Other suitable host cells are known to those skilled in the art, including mouse 3T3 cells as further described in the Examples.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989, the disclosure of which is incorporated herein by reference in its entirety), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an HM74 or HM74a protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce an HM74 or HM74a protein, a homolog thereof, or a fragment of any of the preceding polypeptides. Accordingly, the invention further provides methods for producing a HM74 or HM74a protein using the host cells of the invention. In one embodiment, the method comprises culturing the host cell of invention (into which a recombinant expression vector encoding an HM74 or HM74a protein, a homolog thereof, or a fragment of any of the preceding polypeptides has been introduced) in a suitable medium such that an HM74 or HM74a protein is produced. In another embodiment, the method further comprises isolating an HM74 or HM74a protein, a homolog thereof, or a fragment of any of the preceding polypeptides from the medium or the host cell.

In another embodiment, the invention encompasses a method comprising: providing a cell capable of expressing an HM74 or HM74a polypeptide, or a fragment or homolog thereof, culturing said cell in a suitable medium such that an HM74 or HM74a protein, a homolog thereof, or fragment of any of the preceding polypeptides is produced, and isolating or purifying the HM74 or HM74a protein from the medium or cell.

The host cells of the invention can also be used to produce nonhuman transgenic animals, such as for the study of disorders in which HM74 or HM74a proteins or homologs thereof are implicated. For example, in one embodiment, a host cell of the invention is a fertilized oocyte or an embryonic stem cell into which nucleic acid sequences encoding HM74 or HM74a, a homolog thereof, or a fragment of any of the preceding polypeptides have been introduced. Such host cells can then be used to create non-human transgenic animals in which nucleic acid sequences encoding exogenous HM74 or HM74a, a homolog thereof, or a fragment of any of the preceding polypeptides have been introduced into their genome or homologous recombinant animals in which endogenous nucleic acid sequences encoding HM74 or HM74a, a homolog thereof, or a fragment of any of the preceding polypeptides have been altered. Such animals are useful for studying the expression level of an HM74 or HM74a polypeptide or fragment or homolog thereof and for identifying and/or evaluating modulators of an activity of HM74 or HM74a or homolog thereof. As used herein, a “transgenic animal” is a non-human animal, preferably a mammal, more preferably a rodent such as a rat or mouse, in which one or more of the cells of the animal includes a transgene. Additional examples of transgenic animals include non-human primates, sheep, dogs, cows, goats, chickens, amphibians, and others. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene encoding HM74 or HM74a or homolog thereof has been altered by homologous recombination between the endogenous gene and an exogenous DNA molecule introduced into a cell of the animal, e.g., an embryonic cell of the animal, prior to development of the animal. Methods for generating transgenic animals via embryo manipulation and microinjection, particularly animals such as mice, have become conventional in the art and are described, for example, in U.S. Pat. Nos. 4,736,866 and 4,870,009, both by Leder, et al., U.S. Pat. No. 4,873,191 by Wagner, et al. and in Hogan, B., Manipulating the Mouse Embryo, (Cold Spring Harbor Laboratory Press, Cold Spring Harbor, N.Y., 1986, the disclosures of which are incorporated herein by reference in their entireties).

Preparation of Antibodies to HM74 and/or HM74a

Embodiments of the invention concern antibody compositions, either polyclonal or monoclonal, capable of selectively binding to an HM74 or HM74a polypeptide, a homolog thereof, or a fragment of any of the preceding polypeptides. In certain embodiments, the antibody binds selectively to an epitope-containing a polypeptide comprising a contiguous span of at least 5 amino acids, at least 10 amino acids, at least 15 amino acids, at least 20 amino acids, at least 30 amino acids, at least 40 amino acids, at least 50 amino acids or more than 50 amino acids of an amino acid sequence selected from the group consisting of SEQ ID NOs: 3, 6, and 14. The invention also concerns a purified or isolated antibody capable of specifically binding to a mutated HM74 or HM74a polypeptide or to a fragment, variant, or homolog thereof comprising an epitope of the mutated HM74 or HM74a or homolog polypeptide.

An isolated HM74 or HM74a or homolog polypeptide, or a portion or fragment thereof, can be used as an immunogen to generate antibodies that bind HM74 and/or HM74a or homolog polypeptides using standard techniques for polyclonal and monoclonal antibody preparation.

As used in connection with polypeptides, “fragment” means at least 5 consecutive amino acids, at least 10 consecutive amino acids, at least 15 consecutive amino acids, at least 20 consecutive amino acids, at least 25 consecutive amino acids, at least 30 consecutive amino acids, at least 35 consecutive amino acids, at least 40 consecutive amino acids, at least 45 consecutive amino acids, at least 50 consecutive amino acids, at least 60 consecutive amino acids, at least 70 consecutive amino acids, at least 80 consecutive amino acids, at least 90 consecutive amino acids, at least 100 consecutive amino acids, at least 110 consecutive amino acids, at least 120 consecutive amino acids, at least 130 consecutive amino acids, at least 140 consecutive amino acids, at least 150 consecutive amino acids, at least 160 consecutive amino acids, at least 170 consecutive amino acids, at least 180 consecutive amino acids, at least 190 consecutive amino acids, at least 200 consecutive amino acids, at least 250 consecutive amino acids, at least 300 consecutive amino acids, at least 350 consecutive amino acids or greater than 350 amino acids.

As used in connection with nucleic acids, “fragment” means at least 5 consecutive nucleotides, at least 10 consecutive nucleotides, at least 15 consecutive nucleotides, at least 20 consecutive nucleotides, at least 25 consecutive nucleotides, at least 30 consecutive nucleotides, at least 35 consecutive nucleotides, at least 40 consecutive nucleotides, at least 45 consecutive nucleotides, at least 50 consecutive nucleotides, at least 60 consecutive nucleotides, at least 70 consecutive nucleotides, at least 80 consecutive nucleotides, at least 90 consecutive nucleotides, at least 100 consecutive nucleotides, at least 110 consecutive nucleotides, at least 120 consecutive nucleotides, at least 130 consecutive nucleotides, at least 140 consecutive nucleotides, at least 150 consecutive nucleotides, at least 160 consecutive nucleotides, at least 170 consecutive nucleotides, at least 180 consecutive nucleotides, at least 190 consecutive nucleotides, at least 200 consecutive nucleotides, at least 250 consecutive nucleotides, at least 300 consecutive nucleotides, at least 350 consecutive nucleotides, at least 400 consecutive nucleotides, at least 500 consecutive nucleotides, at least 600 consecutive nucleotides, at least 700 consecutive nucleotides, at least 800 consecutive nucleotides, at least 900 consecutive nucleotides, at least 1000 consecutive nucleotides or greater than 1000 nucleotides.

In some embodiments, a full-length HM74 and/or HM74a or homolog polypeptide can be used as an immunogen. The complete amino acid sequence of human HM74 or HM74a or homolog polypeptide is described in the Sequence Listing.

Alternatively, any fragment of an HM74 or HM74a or homolog polypeptide that contains at least one antigenic determinant may be used to generate antibodies. An antigenic HM74 or HM74a polypeptide fragment comprises at least 5 amino acid residues of an amino acid sequence selected from the group consisting of SEQ ID NOs: 3, 6, and 14 and encompasses an epitope of an HM74 or HM74a or homolog polypeptide such that an antibody raised against the peptide forms a specific immune complex with an HM74 and/or HM74a or homolog polypeptide.

An appropriate immunogenic preparation can contain, for example, a recombinantly expressed HM74 or HM74a or homolog polypeptide or a chemically synthesized HM74 or HM74a or homolog polypeptide. The preparation can further include an adjuvant, such as Freund's complete or incomplete adjuvant, or similar immunostimulatory agent. Immunization of a suitable subject with an immunogenic HM74 or HM74a or homolog polypeptide preparation induces a polyclonal anti-HM74 and/or anti-HM74a or homolog polypeptide antibody response.

An HM74 or HM74a or homolog polypeptide immunogen typically is used to prepare antibodies by immunizing a suitable subject, (e.g., rabbit, goat, mouse or other mammal) with the immunogen. The anti-HM74 and/or anti-HM74a or homolog antibody titer in the immunized subject can be monitored over time by standard techniques, such as with an enzyme linked immunosorbent assay (ELISA) using immobilized HM74 or HM74a or homolog protein. If desired, the antibody molecules directed against HM74 or HM74a can be isolated from the mammal (e.g., from the blood) and further purified by well known techniques, such as protein A chromatography to obtain the IgG fraction. At an appropriate time after immunization, e.g., when the HM74 or HM74a or homolog antibody titers are highest, antibody-producing cells can be obtained from the subject and used to prepare monoclonal antibodies by standard techniques, such as those described in the following references, the disclosures of which are incorporated herein by reference in their entireties: the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an HM74 or HM74a or homolog immunogen as described above, and the culture supernatants of the resulting hybridoma cells are screened to identify a hybridoma producing a monoclonal antibody that binds an HM74 or HM74a or homolog polypeptide.

Any of the many well known protocols used for fusing lymphocytes and immortalized cell lines can be applied for the purpose of generating an anti-HM74 or anti-HM74a, anti-HM74, anti-HM74 homolog or anti-HM74a homolog monoclonal antibody (see, e.g., G. Galfre et al. (1977) Nature 266:55052; Gefter et al. Somatic Cell Genet., cited supra; Lerner, Yale J. Biol. Med, cited supra; Kenneth, Monoclonal Antibodies, cited supra), the disclosures of which are incorporated herein by reference in their entireties. Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind HM74 or HM74a or homolog polypeptide, e.g., using a standard ELISA assay.

Alternative to preparing monoclonal antibody-secreting hybridomas, a monoclonal anti-HM74 or anti-HM74a or anti-homolog antibody can be identified and isolated by screening a recombinant combinatorial immunoglobulin library (e.g., an antibody phage display library) with HM74 or HM74a or a homologous protein to thereby isolate immunoglobulin library members that bind HM74 or HM74a or homolog proteins. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612), the disclosures of which are incorporated herein by reference in their entireties. Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554, the disclosures of which are incorporated herein by reference in their entireties.

Additionally, recombinant anti-HM74 or anti-HM74a or anti-homolog antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060, the disclosures of which are incorporated herein by reference in their entireties.

An anti-HM74 or anti-HM74a or anti-homolog antibody (e.g., monoclonal antibody) can be used to isolate HM74 or HM74a or homologous proteins by standard techniques, such as affinity chromatography or immunoprecipitation. For example, an anti-HM74 or anti-HM74a or anti-homolog antibody can facilitate the purification of natural HM74 or HM74a polypeptides from cells and of recombinantly produced HM74 or HM74a expressed in host cells. Moreover, an anti-HM74 or anti-HM74a or anti-homolog antibody can be used to detect HM74 or HM74a or homolog protein (e.g., in a cellular lysate or cell supernatant) in order to evaluate the abundance and pattern of expression of the HM74 or HM74a or homolog protein. Anti-HM74 or anti-HM74a or anti-homolog antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Upregulation of HM74 and HM74a in Cuboidal Endothelial Cells of Inflamed Tissues

Endothelial cells isolated from inflamed tissue, for example synovial tissue, comprise many cuboidal endothelial cells, which have an expression level of HM74 and/or HM74a that is much increased as compared to cuboidal endothetial cells from non-inflamed tissues and flat (normal) morphology endothelial cells. For example, cuboidal endothelial cells present in inflamed tissue express HM74 and/or HM74a at levels at least about 2-fold greater, at least about 5-fold greater, at least about 10-fold greater, at least about 20-fold greater, at least about 30-fold greater, at least about 40-fold greater, at least about 50-fold greater, at least about 60-fold greater, at least about 70-fold greater, at least about 80-fold greater, at least about 90-fold greater, at least about 100-fold greater, at least about 200-fold greater, at least about 300-fold greater, at least about 400-fold greater, at least about 500-fold greater, at least about 750-fold greater or at least about 1000-fold greater than cuboidal endothetial cells present in non-inflamed tissues and flat morphology endothelial cells.

Cuboidal endothelial cells can be isolated from surrounding tissues by methods described in Example 1. In some embodiments of the methods described herein, isolated cuboidal endothelial cells can be used. However, preparations that are enriched in cuboidal endothelial cells can also be used. In some embodiments of the present invention, unenriched preparations which comprise cuboidal endothelial cells are used.

Primers and Probes

Primers and probes used in the methods described below can be prepared by any suitable method, including, for example, cloning and restriction of appropriate sequences and direct chemical synthesis by a method such as the phosphodiester method of Narang, S. A., et al. (Methods Enzymol, 1979, 68:90-98), the phosphodiester method of Brown, E. L., et al. (Methods Enzymol, 1979, 68:109-151), the diethylphosphoramidite method of Beaucage et al (Tetrahedron Lett, 1981, 22:1859-1862) and the solid support method described in EP 0 707 592, the disclosures of which are incorporated herein by reference in their entireties.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506; and 5,142,047. If desired, the probe may be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in (Urdea, et al. (Nucleic Acids Research. 11:4937-4957, 1988) or Sanchez-Pescador, et al. (J. Clin. Microbiol. 26(10):1934-1938, 1988). In addition, the probes according to the present invention may have structural characteristics such that they allow the signal amplification, such structural characteristics being, for example, branched DNA probes as those described by Urdea, et al. (Nucleic Acids Symp. Ser. 24:197-200, 1991) or in the European patent No. EP 0 225 807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA labeling techniques are well known to the skilled technician.

The probes and primers described herein are useful for a number of purposes. They can be notably used in Southern hybridization to genomic DNA. The probes can also be used to detect PCR amplification products. They may also be used to detect mismatches in an HM74 and/or HM74a or homologous gene or mRNA using other techniques.

Any of the nucleic acids, polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The nucleic acids, polynucleotides, primers and probes of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Any polynucleotide provided herein may be attached in overlapping areas or at random locations on a solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips, and has been generally described in U.S. Pat. No. 5,143,854; PCT publications WO 90/15070 and 92/10092, the disclosures of which are incorporated herein by reference in their entireties.

Identification of Test Compounds which Bind to HM74 and/or HM74a

Some embodiments of the present invention provide a method for identifying modulators, test compounds or agents that bind to HM74 or HM74a or homologous proteins. Other embodiments provide methods for determining whether such compounds have an inhibitory or activating effect on the expression of HM74 and/or HM74a or homolog or the activity level of HM74 and/or HM74a or a homolog thereof. As used herein, “test compound” includes, but is not limited to, synthetic small molecules; natural products; molecules present in tissue, bodily fluid and/or cell extracts; fats; sugars; nucleic acids and polypeptides, such as oligopeptides, antibodies, and peptide domains.

The test compounds can be obtained using any of the numerous approaches in combinatorial library methods known in the art, including: biological libraries; nucleic acid libraries; polypeptide libraries; spatially addressable parallel solid phase or solution phase libraries; synthetic library methods requiring deconvolution; the ‘one-bead one-compound’ library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145, the disclosure of which is incorporated herein by reference in its entirety).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, the disclosures of which are incorporated herein by reference in their entireties.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devin (1990) Science 249:404-406); (Cwirla et al. (1990) Proc. Natl. Acad. Sci. 87:6378-6382); (Felici (1991) J. Mol. Biol. 222:301-310); (Ladner supra.), the disclosures of which are incorporated herein by reference in their entireties.

Alternatively, test compounds can be obtained from a natural products library or from natural extracts, such as biological samples. Examples of biological samples include, but are not limited to, plasma, urine, saliva, lymph, synovial fluid and tissue samples, such as vasculature, muscle and skin.

Assays may be cell based, non-cell-based or in vivo assays. Drug screening assays may be binding assays or expression assays, as further described.

Determining the ability of the test compound to bind to HM74 and/or HM74a or homolog thereof in vitro can be accomplished, for example, by obtaining recominant HM74 and/or HM74a or framents or homologs thereof from an expression system as described above. Although HM74 and/or HM74a or a homolog thereof may be utilized with or without insertion into a membrane, in some embodiments, expressed HM74 and/or HM74a or a homolog thereof is inserted into the plasma membrane of the host cell. In such embodiments, the protein may be solubilized and then further purified. Examples of solubilizing agents include non-ionic detergents such as n-octylglucoside, n-dodecylglucoside, n-dodecylmaltoside, octanoyl-N-methylglucamide, decanoyl-N-methylglucamide, Triton.[RTM. X-100, Triton.RTM. X-114, Thesit.RTM.], Isotridecypoly(ethylene glycol ether)ₙ,3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate. Alternatively, HM74 and/or HM74a or a homolog thereof may be used in its membrane-bound form by preparing microsomes or other light membrane fractions from the expression host.

Once HM74 and/or HM74a or fragments or homologs thereof have been obtained in a suitable form, they are coupled to a radioisotope or enzymatic label such that binding of the HM74 and/or HM74a or fragments or homologs thereof to a test compound can be determined by detecting the labeled HM74 and/or HM74a or fragments or homologs thereof in a complex. For example, HM74 and/or HM74a or fragments or homologs thereof can be labeled with ¹²⁵I, ³⁵S, ¹⁴C, or ³H, either directly or indirectly, and the radioisotope detected by direct counting of radioemmission or by scintillation counting. Alternatively, HM74 and/or HM74a or fragments or homologs thereof can be enzymatically labeled with, for example, horseradish peroxidase, alkaline phosphatase, or luciferase, and the enzymatic label detected by determination of conversion of an appropriate substrate to product. The labeled HM74 and/or HM74a or fragments or homologs thereof is placed in contact with the test compound and the extent of complex formation is measured. For example, the extent of complex formation may be measured by immunoprecipitating the complex or by performing gel electrophoresis.

It is also possible to determine the ability of HM74 and/or HM74a or fragments or homologs thereof to interact with a test compound without the labeling of any of the interactants. For example, a microphysiometer can be used to detect the interaction of a compound with its cognate target molecule without the labeling of either the compound or the target molecule. McConnell, H. M. et al. (1992) Science 257:1906-1912, the disclosure of which is incorporated herein by reference in its entirety. A microphysiometer such as a cytosensor is an analytical instrument that measures the rate at which a cell acidifies its environment using a light-addressable potentiometric sensor (LAPS). Changes in this acidification rate can be used as an indicator of the interaction between compound and cognate target molecule.

Determining the ability of HM74 and/or HM74a or fragments or homologs thereof to bind to a test compound can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705, the disclosures of which are incorporated herein by reference in their entireties. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In some embodiments of the above assay methods, it may be desirable to immobilize either HM74 and/or HM74a or fragments or homologs thereof or the test compound to facilitate separation of complexed from uncomplexed forms of one or both of the molecules, as well as to accommodate automation of the assay. Binding of a test compound to a HM74 and/or HM74a or fragments or homologs thereof, can be accomplished in any vessel suitable for containing the reactants. Examples of such vessels include microtitre plates, test tubes, and micro-centrifuge tubes. In one embodiment, a fusion protein can be provided which adds a domain that allows the protein to be bound to a matrix. For example, glutathione-S-transferase/HM74 and/or HM74a or fragments or homologs thereof can be adsorbed onto glutathione sepharose beads (Sigma Chemical, St. Louis, Mo.) or glutathione derivatized microtitre plates, which are then combined with the test compound and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above.

Other techniques for immobilizing proteins on matrices can also be used in the screening assays of the invention. For example, HM74 and/or HM74a or fragments or homologs thereof can be immobilized utilizing conjugation of biotin and streptavidin. Biotinylated HM74 and/or HM74a or fragments or homologs thereof can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with an HM74 and/or HM74a or fragments or homologs thereof but which do not interfere with binding of the HM74 and/or HM74a or fragments or homologs thereof to the test compound can be derivatized to the wells of the plate, and unbound HM74 and/or HM74a or fragments or homologs thereof trapped in the wells by antibody conjugation. Methods for detecting such complexes, in addition to those described above for the GST-immobilized complexes, include immunodetection of complexes using antibodies reactive with the HM74 and/or HM74a or fragments or homologs thereof, as well as enzyme-linked assays which rely on detecting an enzymatic activity associated with HM74 and/or HM74a or fragments or homologs thereof.

In one embodiment, the screening assay comprises contacting a cell which expresses HM74 and/or HM74a or fragments or homologs thereof, with test compound and the binding interaction is detected. In such cases, the test compound can be labeled to facilitate detection of binding. Alternatively, binding can be determined by evaluating the ability of HM74 and/or HM74a or fragments or homologs thereof to modulate the activity of a downstream effector, for example, generating a signal in the GPCR transduction pathway.

In screening assays where cells or portions of cells are used, the HM74 and/or HM74a can be produced recombinantly or naturally in a cell having an increased expression of HM74 and/or HM74a. We have discovered that expression of HM74 and HM74a is specifically upregulated in cuboidal endothelial cells obtained from inflamed tissues. The expression levels of HM74 and HM74a these cuboidal endothelial cells is substantially increased when compared with the expression levels in endothelial cells from non-inflamed tissue. As such, in a preferred embodiment, cuboidal endothetial cells expressing HM74 and/or HM74a are used to screen test compounds to identify those compounds that bind to HM74 and/or HM74a.

In some embodiments, the HM74 or HM-74a proteins, fragments thereof, or homologs thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins or petpides, which bind to or interact with HM74, HM74a, fragments thereof, or homologs thereof. Such HM74, HM74a, or homolog-binding proteins or peptides may be involved in the propagation of signals by the HM74, HM74a or homologous proteins. For example, such proteins or peptides may activate or inhibit the HM74, HM74a, or homologous protein or may act as downstream elements of signaling pathway mediated by the HM74, HM74a or homologous protein.

The two-hybrid system is based on the modular nature of most transcription factors, which consist of separable DNA-binding and activation domains. Briefly, the assay utilizes two different DNA constructs. In one construct, the gene that codes for a HM74, HM74a or homologous protein or a fragment thereof is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the HM74, HM74a or homologous protein.

Identification of Test Compounds which Regulate the Level of Activity or Expression of HM74 and/or HM74a

Test compounds that bind to HM74 and/or HM74a or fragments or homologs thereof are then assayed to determine whether they reduce the level of activity or expression level of HM74 and/or HM74a. In one embodiment, endothelial cells from inflamed tissues are used to determine whether the test compound is capable of reducing the level of activity or expression level of HM74 and/or HM74a. In such embodiments, the level of activity or the level of expression of HM74 and/or HM74a is measured in endothelial cells from both inflamed and non-inflamed (control) tissues. The endothelial cells from both inflamed and control tissues are then incubated with the test compound. After the incubation, the level of activity or the level of expression of HM74 and/or HM74a is again measured for both the inflamed and control cells. After contact with the test compound, if the level of activity or level of expression of HM74 and/or HM74a or a homolog thereof has decreased in the endothelial cells from inflamed tissues, the test compound is effective at reducing the expression or activity level of HM74 and/or HM74a or a homolog thereof. Since increased expression or activity of HM74 and/or HM74a or a homolog thereof is associated with progression of inflammatory disorders, the reduction in expression or activity of HM74 and/or HM74a or a homolog thereof can lead to a reduction in symptoms associated with an inflammatory disorder.

Methods for detecting the level of expression of HM74 and/or HM74a or homologs thereof include methods of detecting the transcripts encoding HM74 and/or HM74a or homologous polypeptides or the polypeptides themselves (HM74 gene products). For example, HM74 and/or HM74a or homologs thereof can be detected by immunocytochemistry using a labeled antibody that binds to HM74 and/or HM74a or a homolog thereof. The preparation of such antibodies has been describe above. Measuring the intensity of staining by immunohistochemistry also provides a technique by which to determine relative expression levels of a gene product. Alternative immunohistochemical methods can be used to determine the relative amounts of HM74 and/or HM74a or homolog mRNA present in cells. Such methods are exemplfied in Example 2 below.

A quantitative method for determining the amount of gene product expression for HM74 and/or HM74a or a homolog thereof is by quantitative PCR (Q-PCR). Various methods of Q-PCR are known in the art. Q-PCR can be used to measure the amount of mRNA that is produced by a cell at any point in time. For example, Q-PCR can be used to determine the expression level of HM74 and/or HM74a or homolog mRNA in cells both prior and subsequent to the incubation of the cells with the test compound. Since Q-PCR is quantitative, the level of HM74 and/or HM74a or homolog expression before and after contact of the cell with the test compound can easily be determined.

Additionally, test compounds that bind to HM74 and/or HM74a or fragments or homologs thereof can be further assayed in animal models of inflammation to determine whether they are effective at reducing symptoms associated with inflammatory disorders. As used herein, the phrase “is effective at reducing” or “effectively reduces” means that a substance is capable of decreasing one or more symptoms associated with a condition or disorder by a detectable amount.

One preferred animal model of inflammatory disease is the mouse model of rheumatoid arthritis. In this model, RA is induced in mice using collagen. In some embodiments, mice are prepared by immunizing with collagen then providing a booster about three weeks after the initial immunization. To evaluate the effect of the test compound, it is administered to the mice on a fixed schedule. For example, the mice may be treated teated daily with the test compound via IP injections. The test compound can be evaluated at doses up to the level where the compound displays substantial toxicity. For example, the test compound can be administered at dose of 150, 50, 15, and 5 μg/day. Similar doses of a control compound should be administered to a control group of mice. The incidence and severity of arthritis is then monitored in a blind study. For example, each paw is assigned a score from 0 to 4 as follows: 0=normal; 1=swelling in 1 to 3 digits; 2=mild swelling in ankles, forepaws, or more than 3 digits; 3=moderate swelling in multiple joints; 4=severe swelling with loss of function. Each paw is totaled for a cumulative score/mouse. The cumulative scores are then totaled for mice in each group for a mean clinical score.

Once obtained, the mean clinal score is used to determine whether the test compound effectively reduces the symptoms associated with the inflammatory disorder. An decrease in signs of symptoms in the treatment group as compared to the control group indicates that the test compound is effective in reducing the symptoms associated with an inflammatory disorder.

Amelioration of Inflammatory Disorders

The methods of amelioration of inflammatory disorders involve reducing the expression level of HM74 and/or HM74a or a homolog thereof in the cells of inflamed tissues. In one embodiment, the expression level or activity level of HM74 and/or HM74a or a homolog thereof is reduced by providing a compound that effectively inhibits expression or activity of the HM74 and/or HM74a or homolog gene product. In an alternative embodiment, the expression level or activity level of HM74 and/or HM74a or a homolog thereof is reduced by providing a compound that causes a downregulation of this receptor. Additionally, there are a number of other mechanisms by which a compound can reduce the expression level or activity level of HM74 and/or HM74a or a homolog thereof in an inflamed tissue. The expression level or activity level of HM74 and/or HM74a or a homolog thereof can be reduced by any of these mechanisms.

It will be appreciated that “ameliorating” refers to clinical intervention in an attempt to alter the natural course of the individual being treated, and may be performed either for prophylaxis or during the course of clinical pathology. Ameliorating a disorder does not necessarily require that the disorder be completely cured. Desirable effects include preventing occurrence or recurrence of disease, alleviation of symptoms, diminishment of any direct or indirect pathological consequences of the disease, such as lowering the rate of disease progression, amelioration or palliation of the disease state, and remission or improved prognosis. The “pathology” associated with a disease condition is anything that compromises the well-being, normal physiology, or quality of life of the affected individual.

In some embodiments of the present invention, amelioration is performed by administering to an individual an effective amount of a compound that reduces the expression or activity of HM74 and/or HM74a or a homolog thereof. An “effective amount” is an amount sufficient to effect a beneficial or desired clinical result, and can be administered in one or more doses. An “individual” treated by the methods of this invention is a vertebrate, particularly a mammal (including model animals of human disease, farm animals, sport animals, and pets), and typically a human.

In some embodiments of the present invention, amelioration of a disorder, such as an inflammatory disorder, is mediated by administering a compound that reduces the expression level or activity level of HM74 and/or HM74a or a homolog thereof, wherein the compound is formulated as a pharmaceutical composition. Such compounds can be incorporated into pharmaceutical compositions suitable for administration to humans. Pharmaceutical compositions typically comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions described herein are formulated to be compatible with the intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELa (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Where the active compound is a protein, peptide or anti-HM74 and/or anti-Hm74a or anti-homolog antibody, sterile injectable solutions can be prepared by incorporating the active compound (e.g.,) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Most preferably, active compound is delivered to a subject by intravenous injection.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, the disclosure of which is incorporated herein by reference in its entirety.

It is especially advantageous to formulate oral or preferably parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Detection of Expression of HM74 and/or HM74a or a Homolog Thereof in Cuboidal Cells

An exemplary method for detecting the presence (quantitative or not) or absence of an HM74 and/or HM74a gene product (protein or nucleic acid) in a biological sample involves obtaining a biological sample from an individual or test subject and contacting the biological sample with a compound or an agent capable of detecting an HM74 and/or HM74a gene product or a homolog thereof such that the presence of the HM74 and/or HM74a gene product or a homolog thereof is detected in the biological sample. A preferred agent for detecting an HM74 and/or HM74a gene or mRNA or a homolog thereof is a labeled nucleic acid probe capable of hybridizing to an HM74 and/or HM74a gene or mRNA or a homolog thereof. The nucleic acid probe can be, for example, a full-length HM74 and/or HM74a nucleic acid or a shorter nucleic acid of at least about 10, 15, 30, 50, 100, 250, 400, 500 or 1000 nucleotides in length and sufficient to specifically hybridize under moderate conditions to an HM74 and/or HM74a genomic DNA or mRNA or a homolog thereof or portions of an HM74 and/or HM74a genomic DNA or mRNA or a homolog thereof. Other suitable probes for use in the diagnostic assays of the invention are described herein.

An alternative agent for detecting an HM74 and/or HM74a gene product or a homolog thereof is an antibody capable of binding to an HM74 and/or HM74a gene product or a homolog thereof, preferably an antibody with a detectable label. Antibodies can be polyclonal, or more preferably, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection methods described herein can be used to detect an HM74 and/or HM74a gene product or a homolog thereof in a biological sample in vitro as well as in vivo. For example, in vitro techniques for detection of an HM74 and/or HM74a mRNA or a homolog thereof include, but are not limited to, Northern hybridizations and in situ hybridizations. In vitro techniques for detection of an HM74 and/or HM74a protein or a homolog thereof include, but are not limited to, enzyme linked immunosorbent assays (ELISAs), Western blots, immunoprecipitations and immunofluorescence. In vitro techniques for detection of an HM74 and/or HM74a genomic DNA include, but are not limited to, Southern hybridizations. Furthermore, in vivo techniques for detection of an HM74 and/or HM74a protein or a homolog thereof include introducing into a subject a labeled anti-HM74 and/or anti-HM74a or anti-homolog antibody. For example, the antibody can be labeled with a radioactive marker whose presence and location in a subject can be detected by standard imaging techniques.

Some embodiments of the present invention provide diagnostic, prognostic and monitoring assays for detecting the level of expression of HM74 and/or HM74a or a homolog thereof in inflamed tissues. Methods for detecting the expression and/or determining the expression level of an HM74 and/or HM74a gene product or a homolog thereof include, but are not limited to, methods based on immunohistochemistry and methods based on Q-PCR. However, it will be appreciated that any method which provides for specific or semi-specific detection of a molecule such as HM74 and/or HM74a or a homolog thereof can be used with the diagnostic and monitoring assays described herein.

In some embodiments of the present invention the expression or the expression level of HM74 and/or HM74a or a homolog thereof is determined in cells present in a biological sample of inflamed tissue. Biological samples can be collected by any of the methods well known in the art. In some embodiments of the present invention, the biological sample is inflamed synovial tissue. In other embodiments, the tissue is non-inflamed synovial tissue. Tissue may be taken from individuals known to suffer from RA, such as in the case of monitoring and prognostic applications. Alternatively, tissue may be taken from individuals not known to be suffering from RA, such as in the case of diagnostic applications.

Biological samples, such as synovial tissue, may be used without further treatment; however, in some embodiments of the present invention, the biological samples are prepared so as to enrich or isolate one or more specific cell populations. In some embodiments, enriched preparations of endothelial cells are prepared from the biological samples. In other embodiments, preparations enriched in cuboidal endothelial cells are prepared. Methods for preparing enriched or isolated populations of cubodial endothelial cells from a biological sample are described in Example 1.

In diagnostic applications, a biological sample from an individual, such as synovial tissue, can be tested to determine whether the individual suffers from an inflammatory disorder, such as RA. The biological sample is obtained by methods known in the art. In some embodiments, the biological sample is further prepared to enrich one or more specific cell types, such as endothelial cells, as described above. Cells of the biological sample are then tested, for example, using a method such as immunohistochemistry or Q-PCR, to detect expression of HM74 and/or HM74a or a homolog thereof. Since very little or no HM74 and/or HM74a is expressed in normal endothelial cells from non-inflamed tissues and since the level of expression of HM74 and/or HM74a is increased in cells of inflamed tissues, the expression of HM74 and/or HM74a in the biological sample is diagnostic for an inflammatory disorder. As described below, the level of HM74 and/or HM74a expression in cells of inflamed tissues can provide infromation about the severity of the inflammatory disorder.

In some diagnostic embodiments, the expression of HM74 and/or HM74a or a homolog thereof is examined in the cells of a biological sample obtained from an individual who is known not to suffer from an inflammatory disorder. Such a biological sample can be used as a control tissue by which to establish a baseline for HM74 and/or HM74a or homolog expression in cells of non-inflamed tissue.

In view of the observation that the amount of HM74 and/or HM74a expression correlates to the severity of an inflammatory disorder, some embodiments of the present invention relate to methods of monitoring the severity of an inflammatory disorder by determining whether the level of HM74 and/or HM74a expression is increased in a biological sample of inflamed tissue comprising cuboidal endothelial cells relative to the level of HM74 and/or HM74a expression in cells of non-inflamed tissue. In such embodiments, the level of HM74 and/or HM74a is measured by methods such as immunohistochemistry or Q-PCR. The level of HM74 and/or HM74a expressed in cells of inflamed tissue relative to the level of HM74 and/or HM74a expressed in non-inflamed tissue is determined so as to determine how far the disease has progressed.

In other embodiments, the level of expression of HM74 and/or HM74a may be used to monitor the effectiveness of treatment of an inflammatory condition, such as RA. In these embodiments a biological sample of inflamed tissue comprising cuboidal endothelial cells is obtained before the beginning of the treatment regimen and at various time points throughout the treatment regimen. The levels of expression of HM74 and/or HM74a in the cells of the samples are comprared to determine the effectiveness of the treatment. If the treatment is effective, the level of HM74 and/or HM74a will decrease during the course of the treatment.

### EXAMPLES

Having generally described this invention, a further understanding can be obtained by reference to certain specific examples which are provided herein for purposes of illustration only, and are not intended to be limiting unless otherwise specified.

### Example 1

### Isolation of Synovial Endothelial Cells from Inflammed Synovial Tissues

Synovial tissues specimens were obtained from RA patients undergoing joint replacement surgery as a normal part of their clinical care. All included patients satisfied the American College of Rheumatology criteria for the diagnosis of RA. Synovium was cut into small pieces with scissors, and washed over a steel screen to remove lymphocytes before enzymatic digestion for 90 min at 37° C. in RPMI 1640 containing 2 mg/ml collagenase A, 0.04 mg/ml Dnase, 50 μg/ml gentamicin and 0.25 μg/ml amphotericin-B. The cell suspension successively was passed through 74 and 40 μm stainless steel meshes. The final eluate consisted mainly of single cells and capillary loops with little contaminating debris. To facilitate endothelial (EC) selection from the stroma cells suspension without any cell culture step, we then performed two steps of leukocyte depletion with specific antibodies (CD3, CD19, CD45 and CD14) coupled to Dynabeads (Dynal France, Compiègne, France), followed by one step of erythrocytes depletion with monoclonal antibody anti-Glycophorin A. Synovial ECs were subsequently selected using the MACS system (Myltenyi Biotec, Paris, France). The cells were labelled with mouse Ab anti-human DARC (Duffy Antigen Receptor for Chemokine) for 15 minutes at 4° C., followed by goat anti-mouse antibody conjugated to MACS superparamagnetic microbeads for 10 minutes at 4° C., and finally Cy3-labelled donkey anti-mouse IgG (H+ L) (Jackson Immunoresearch, West Grove, Pa.) for 5 minutes at 4° C. DARC positive ECs were then isolated by multiple rounds of selection (1× to 4×) on a MS MACS column according to the manufacturer's instructions. Synovial ECs were finally eluted and counted on a Neubauer Slide under a fluorescence microscope in order to assess the final cell number and purity. Cell preparations passing the quality control (QC) threshold were centrifugated and immediately placed in the lysis buffer from Strataprep Total RNA Microprep Kit (Stratagene, La Jolla, Calif.) for RNA extraction under conditions described by manufacturer.

### Example 2

### The Expression and Tissue Distribution of HM74 and HM74a in Inflammed Tissues

This Example describes the analysis of the expression and tissue distribution of HM74 and HM74a transcripts by in situ hybridization in inflammed tissue samples and control samples.

In situ hybridization (ISH) analysis of HM74 and HM74a mRNA expression in Crohn's disease (CD) and rheumatoid arthritis (RA) was performed following the non-radioactive ISH method originally described by B. St Croix, et al. (Science 18, no 289,1197-202, 2000) with minor modifications. Briefly, antisense and sense HM74 riboprobes were generated by PCR by incorporating the T7 promoter sequence (5′GGATCCTAATACGACTCACTATAGGGAGA3′) (SEQ ID NO: 15) into the forward or reverse HM74 primer, respectively 5′GTGTGCATCAGCTTCAGCAT3′ (SEQ ID NO: 16) and 5′GAGGCAGATGTGGGAAGAAG3′ (SEQ ID NO: 17). PCR was performed on a plasmid template containing the complete coding sequence of HM74 (pcDNA3.1-HM74) using conventional procedures. PCR amplification was verified by gel electrophoresis before performing in vitro transcription with Dig-labelled UTP on 200 ng of PCR product with the DIG RNA labelling mix (Roche) following the manufacturer's instructions. RNA integrity and concentration was verified by running 5 μl of RNA on a 6% TBE-urea gel along side known concentrations of marker (RNA century-Plus size markers, Ambion).

Frozen sections from biopsies of patients with RA and CD were prepared according to conventional histological procedures. After fixation in 4% paraformaldehyde/PBS for 1 hour, sections were rinsed twice in PBS for 5 min on ice and then incubated with 0.2N HCl, 5 min at room temperature (RT) to inactivate endogenous alkaline phosphatase activity. Sections were then immediately incubated in 2% Dako's ready to use Pepsin for 5 mins at 37° C. before rinsing for 5 min on ice with PBS.

Slides were acetylated in 0.1 M Triethylamine (pH 8.0) for 5 mins, rinsed for 5 mins in PBS and equilibrated for 5 to 10 mins in 5×SSC. The acetylated slides were removed from SSC and a CoverWell incubation chamber gasket (Molecular Probes cat#18156) was placed around a section of the slide for the prehybridization step. Briefly, 150 μl of mRNA hybridization buffer (Dako cat#S3304) was added to the chamber and allowed to incubate 1 hour at 55° C. In the meantime, digoxigenin-labeled riboprobe was added in a 1.5 ml RNAse-free microfuge tube to a final concentration of 200 ng/ml, denatured at 95° C. for 3 mins and chilled immediately on ice.

The hybridization step was performed by adding 150 μl of denaturated, labelled riboprobes to the tissue section contained in the chamber. After sealing the chamber with a coverslip, slides were placed in a humid box and incubated overnight at 55° C. The next day, the slides were rinsed by incubating in 50 ml tubes containing 30 ml 2×SSC for 5 min in a 45° C. water bath. The slides were then rinsed twice in TNE buffer at 45° C. for 5 min. Excess unhybridized riboprobe was removed by incubation in 250 ul RNAse A/T1 cocktail (Ambion cat# 2288) diluted 1/35 in TNE buffer at 37° C. for 1 hour. Slides were then stringently washed twice with 30 ml 2×SSC, 50% deionized formamide for 20 min at 55° C. and then rinsed once with 30 ml 0.08×SSC for a further 20 min at 55° C.

The following methods use conventional procedures and reagents classically used for the detection of non-radioactive nucleic acids using the biotin-tyramide amplification cycle system (Dako). Briefly, slides were rinsed with 1×TBST buffer for 3 min at RT and incubated with 150 μl of blocking buffer containing 1/20 dilution of rabbit immunoglobulin fraction (Dako cat#X0903) for 30 min at RT. Sections were then incubated with HRP-anti-DIG (Dako cat#P5104) diluted 1:150 in bloking buffer for 45 min at RT and washed three times for 4 min with 1×TBST buffer prior to adding one drop of ready-to-use biotinyl-tyramide (Dako GenPoint Kit). The slides were further incubated in dark for 8 min at RT. The slides were then rinsed three times for 4 min with 1×TBST buffer, incubated with rabbit HRP-anti-biotin (Dako) diluted 1:150 in bloking buffer for 20 min at RT and rinsed again as above prior to the addition of a second drop of ready-to-use biotinyl-tyramide (Dako GenPoint Kit). The slides were further incubated in dark for 5 min at RT. After washing three times for 4 mins with 1×TBST buffer at RT, slides were incubated with rabbit AP-anti-biotin (Dako) diluted 1:75 with blocking buffer for 20 min at RT in dark. After a last washing step, specific signal detection was performed by incubating the AP-substrate (Fast Red tablets, Sigma) for 20 min at RT in dark. The signal was carefully monitored under a fluorescent microscope (Leica) until specific fluorescence signal appeared. Specific fluorescence was determined by comparing the signal to the background observed on a control slide. Reactions were stopped by incubating slides in water for 3 min at RT. Sections were allowed to dry for 5 min at RT and mounted after adding one drop of Supermount permanent aqueous mounting media (Biogenex). An extra immunostaining step was processed for some slides and followed conventional immunostaining procedures to obtain dual-color labelling pictures.

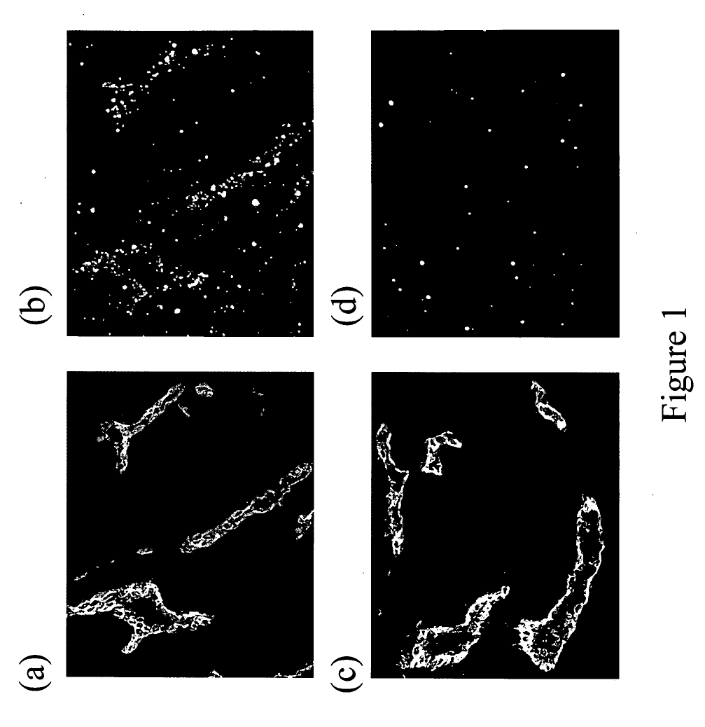

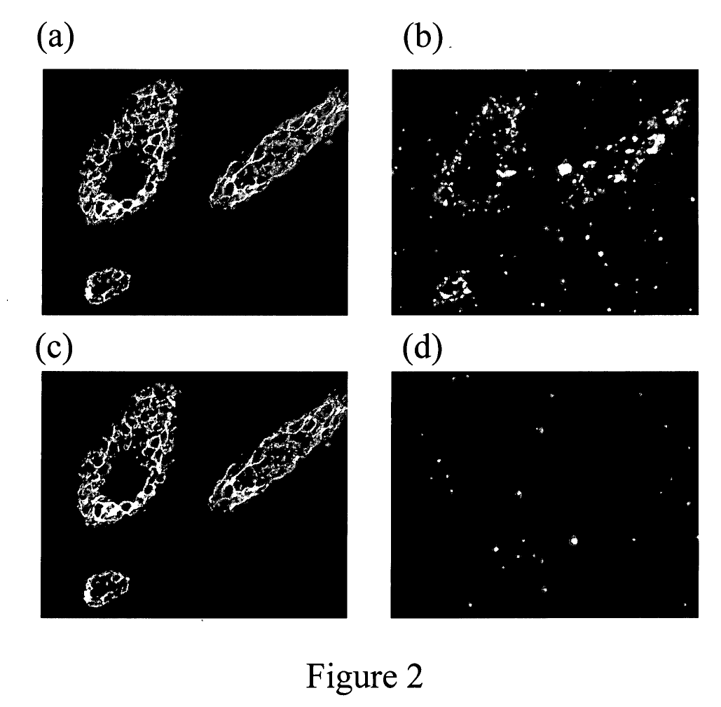

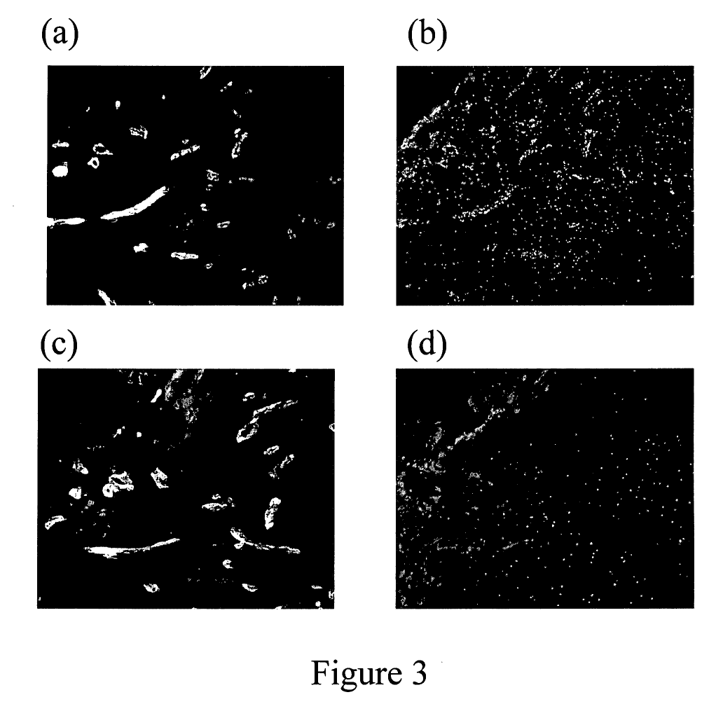

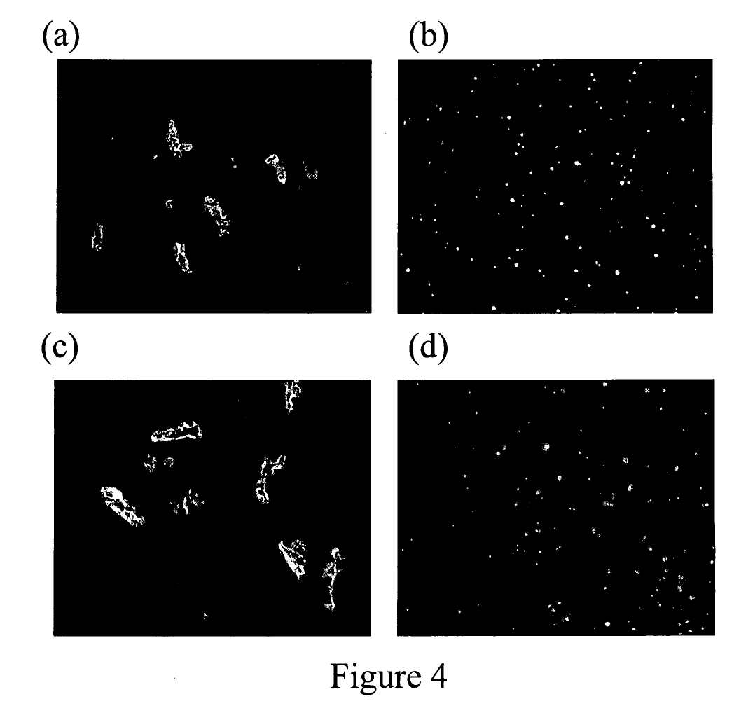

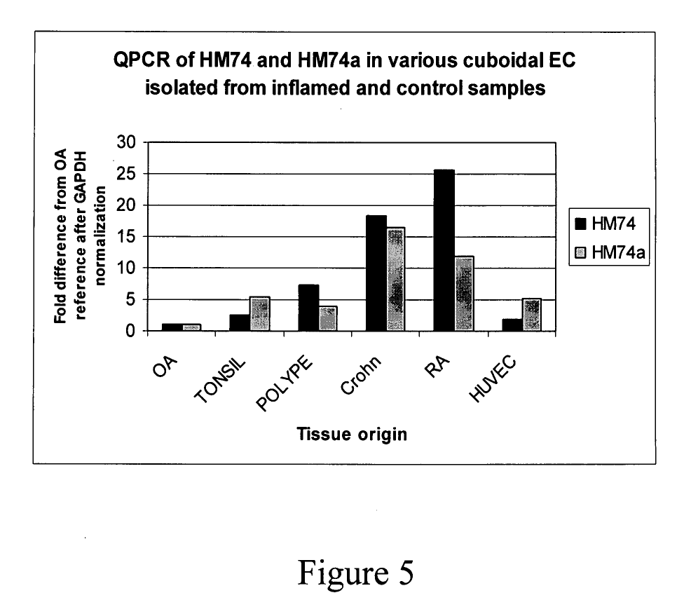

These ISH analyses revealed a strong and specific expression of HM74 mRNA in endothelial cells from small blood vessels in RA disease (FIGS. 1A-D), in CD (FIGS. 2A-D) and in human inflammed tonsil (FIGS. 3A-D) as observed after signal co-localization of HEV-like vessels endothelial cells labeled by immunohistochemistry (IHC) with anti-DARC antibody (see, FIGS. 1A, 1C, 2A, 2C, 3A and 3C). The intensity of the signals in microvascular endothelial cells was consistently greater in inflamed tissues compared to that of in EC of tissues with a weaker synovitis score. The signal was not detected in endothelial cells from non-inflamed tissues or in normal appearing tissues (see, FIGS. 4 and 5).

### Example 3

### Transcription of HM74 and HM74a mRNAs in Inflammed Tissues

This Example describes up-regulation of HM74 and HM74a expression in primary human endothelial cells freshly isolated from RA patients. In particular, HM74 and HM74a mRNA expression in primary human endothelial cells from different inflamed and non-inflamed tissues were analysed using real time quantitative RT-PCR.

Total RNA from RA and non-RA patients (non-inflamed OA) were isolated from 500,000 cells with the Absolutely RNA microprep (Stratagene, La Jolla, Calif., USA) according to the manufacturer's instructions. RNA preparations (20 ng of each) were reverse transcribed and amplified for 21 cycles with a Super-SMART-PCR cDNA synthesis kit (Clontech, Palo Alto, Calif., USA) according to the manufacturer's instructions. Amplified cDNA concentrations were determined from absorbance measurements by spectrophotometry.

Real-time PCR was conducted on 5 ng of Super-SMART cDNA and 0.4 μM of each primer using SYBR Green PCR Master Mix kit (Applied Biosystems, Foster City, Calif., USA) in a final volume of 25 μl. Specific PCR primers were HM74 forward 5′GCCCAACCTCAAATAACCATT3′ (SEQ ID NO: 18), HM74 reverse 5′TCTCTGGCTCCAACTCTGCT3′ (SEQ ID NO: 19), HM74a forward 5′CTGGGCCCAACCTCTCCTT3′ (SEQ ID NO: 20), HM74a reverse 5′GCAAAAGTTTCAGATGCCTAGAAG3′ (SEQ ID NO: 21), GAPDH forward 5′GAGTCAACGGATTTGGTCGT3′ (SEQ ID NO: 22), and GAPDH reverse 5′GACAAGCTTCCCGTTCTCAG3′ (SEQ ID NO: 23). The housekeeping gene GAPDH was evaluated in all samples as an internal control. The cycling conditions were as follows: initial denaturation at 95° C. for 10 min, followed by 40 cycles of 95° C. for 10 sec and 60° C. for 1 min. PCR products (10 ul) were separated on a 2% agarose/ethidium bromide gel for visualization. Reactions were analysed with AB17700 Prism SDS sequence detection system (Applied Biosystems, Foster City, Calif., USA) and the threshold cycles (Ct) for each sample run in duplicate were determined. The relative expression of HM74 and HM74a in endothelial cells was calculated using the 2(-Delta Delta C(T)) method, as previously described (Livak, K. J. and T. D. Schmittgen. 2001. Methods. 25:402.).

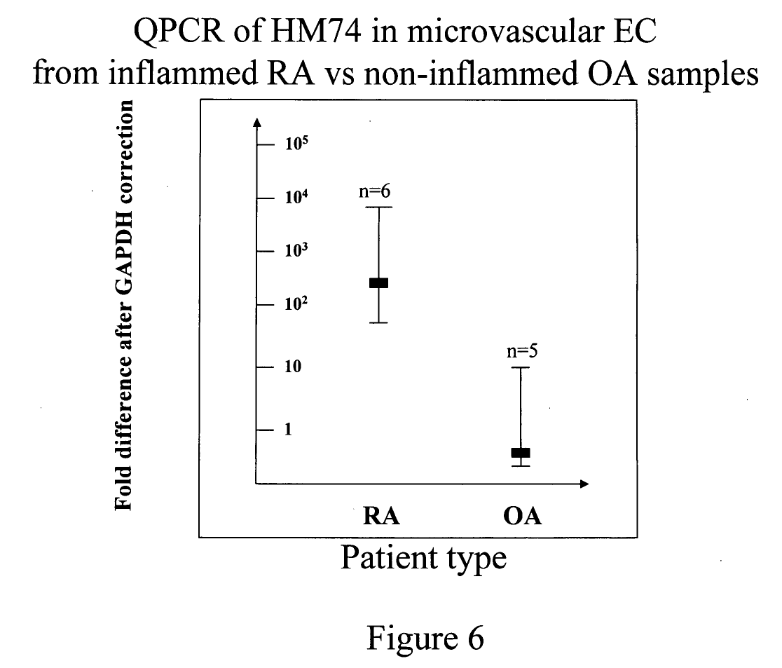

These quantitative RT-PCR analyses revealed that HM74 and HM74a mRNA expressions were strongly induced (10-fold) in all types of primary human endothelial cells from chronic inflammatory tissues (FIG. 5), particularly in cuboidal endothelial cells from RA patients (FIG. 6).

The methods described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. Accordingly, it will be apparent to one skilled in the art that varying substitutions and modifications may be made to the invention disclosed herein without departing from the scope and spirit of the invention.

The disclosures of all references cited herein are expressely incorporated herein by reference in their entireties.

What is claimed is:

1. A method of detecting the expression of a gene product, said method comprising:
obtaining a cuboidal endothelial cell; and
determining whether one or more gene products selected from the group consisting of HM74, HM74a and homologs thereof is expressed in said cuboidal endothelial cell.

2. The method of claim 1, wherein the expression of one or more gene products selected from the group consisting of HM74, HM74a and homologs thereof is determined by quantitative polymerase chain reaction (Q-PCR).

3. The method of claim 1, wherein the expression of one or more gene products selected from the group consisting of HM74, HM74a and homologs thereof is determined by a binding a detector reagent thereto.

4. The method of claim 3, wherein the said detector reagent is an antibody.

5. The method of claim 1, wherein said one or more gene products is HM74.

6. The method of claim 1, wherein said one or more gene products is HM74a.

7. A method of monitoring the severity of an inflammatory disorder, said method comprising the steps of:
obtaining a biological sample of an inflamed tissue, said biological sample comprising cuboidal endothelial cells;
measuring the expression level of one or more gene products in said cuboidal endothelial cells, wherein said one or more gene products is selected from the group consisting of HM74, HM74a and homologs thereof; and
determining whether the expression level of said one or more gene products selected from the group consisting of HM74, HM74a and homologs thereof in said cuboidal endothelial cells is increased relative to the expression level of said one or more gene products selected from the group consisting of HM74, HM74a and homologs thereof in endothelial cells present in a non-inflamed tissue, wherein an increased expression level of said one or more gene products selected from the group consisting of HM74, HM74a and homologs thereof in said cuboidal endothelial cells indicates increased severity of said inflammatory disorder.

8. The method of claim 7 further comprising isolating endothelial cells from said biological sample.

9. The method of claim 7, wherein said inflammatory disorder is rheumatoid arthritis.

10. The method of claim 7, wherein said biological sample is synovial tissue.

11. The method of claim 7, wherein said expression level of said one or more gene products selected from the group consisting of HM74, HM74a and homologs thereof is measured by Q-PCR.

12. The method of claim 7, wherein said expression level of said one or more gene products selected from the group consisting of HM74, HM74a and homologs thereof is measured by immunohistochemistry.

13. The method of claim 7, wherein said determining step comprises determining whether said expression level of said one or more gene products selected from the group consisting of HM74, HM74a and homologs thereof in cuboidal endothelial cells is at least about 2-fold greater than the expression of said one or more gene products selected from the group consisting of HM74, HM74a and homologs thereof in endothelial cells from non-inflamed tissue.

14. The method of claim 7, wherein said determining step comprises determining whether said expression level of said one or more gene products selected from the group consisting of HM74, HM74a and homologs thereof in cuboidal endothelial cells is at least about 10-fold greater than the expression of said one or more gene products selected from the group consisting of HM74, HM74a and homologs thereof in endothelial cells from non-inflamed tissue.

15. The method of claim 7, wherein said determining step comprises determining whether said expression level of said one or more gene products selected from the group consisting of HM74, HM74a and homologs thereof in cuboidal endothelial cells is at least about 20-fold greater than the expression of said one or more gene products selected from the group consisting of HM74, HM74a and homologs thereof in endothelial cells from non-inflamed tissue.

16. The method of claim 7, wherein said determining step comprises determining whether said expression level of said one or more gene products selected from the group consisting of HM74, HM74a and homologs thereof in cuboidal endothelial cells is at least about 50-fold greater than the expression of said one or more gene products selected from the group consisting of HM74, HM74a and homologs thereof in endothelial cells from non-inflamed tissue.

17. The method of claim 7, wherein said determining step comprises determining whether said expression level of said one or more gene products selected from the group consisting of HM74, HM74a and homologs thereof in cuboidal endothelial cells is at least about 100-fold greater than the expression of said one or more gene products selected from the group consisting of HM74, HM74a and homologs thereof in endothelial cells from non-inflamed tissue.

18. The method of claim 7, wherein said one or more gene products is HM74.

19. The method of claim 7, wherein said one or more gene products is HM74a.

20. A method of diagnosing an inflammatory disorder, said method comprising:
obtaining a biological sample of an inflamed tissue, said biological sample comprising cuboidal endothelial cells;
determining whether one or more gene products selected from the group consisting of HM74, HM74a and homologs thereof is expressed in said cuboidal endothelial cells, wherein the presence of said one or more gene products selected from the group consisting of HM74, HM74a and homologs thereof in said cuboidal endothelial cells is indicative of an inflammatory disorder.

21. The method of claim 20, wherein said inflammatory disorder is rheumatoid arthritis.

22. The method of claim 20, wherein said biological sample is synovial tissue.

23. The method of claim 20, wherein Q-PCR is used to determine whether one or more gene products selected from the group consisting of HM74, HM74a and homologs thereof is expressed in said cuboidal endothelail cells.

24. The method of claim 20, wherein immunohistochemistry is used to determine whether one or more gene products selected from the group consisting of HM74, HM74a and homologs thereof is expressed in said cuboidal endothelail cells.

25. The method of claim 20, wherein said one or more gene products is HM74.

26. The method of claim 20, wherein said one or more gene products is HM74a.

27. A method of evaluating whether a test compound is effective at reducing the symptoms of an inflammatory disorder, said method comprising the steps of:
determining the expression level of one or more gene products selected from the group consisting of HM74, HM74a and homologs thereof in a cuboidal endothelial cell;
contacting said cuboidal endothelial cell with a test compound;
determining the expression level of said one or more gene products selected from the group consisting of HM74, HM74a and homologs thereof in said cuboidal endothelial cell that has been contacted with said test compound;
determining whether the expression level of said one or more gene products selected from the group consisting of HM74, HM74a and homologs thereof in said cuboidal endothelial cell that has been contacted with said test compound is increased relative to the expression level of said one or more gene products selected from the group consisting of HM74, HM74a and homologs thereof in said cuboidal endothelial cell that has not been contacted with said test compound, wherein a reduced expression level of said one or more gene products selected from the group consisting of HM74, HM74a and homologs thereof in said cuboidal endothelial cell that has been contacted with said test compound relative to the expression level of said one or more gene products selected from the group consisting of HM74, HM74a and homologs thereof in said cuboidal endothelial cell that has not been contacted with said test compound indicates that said test compound is effective at reducing the symptoms of an inflammatory disorder.

28. The method of claim 27, wherein said inflammatory disorder is rheumatoid arthritis.

29. The method of claim 27, wherein said expression level of said one or more gene products selected from the group consisting of HM74, HM74a and homologs thereof is measured by Q-PCR.

30. The method of claim 27, wherein said expression level of said one or more gene products selected from the group consisting of HM74, HM74a and homologs thereof is measured by immunohistochemistry.

31. The method of claim 27, wherein said one or more gene products is HM74.

32. The method of claim 27, wherein said one or more gene products is HM74a.

33. A method of evaluating whether a test compound is effective at reducing the symptoms of an inflammatory disorder, said method comprising determining whether said test compound reduces the expression level of one or more gene products selected from the group consisting of HM74 and HM74a in cuboidal endothelial cells.

34. The method of claim 33, wherein said inflammatory disorder is rheumatoid arthritis.

35. The method of claim 33, wherein said expression level of said one or more gene products selected from the group consisting of HM74, HM74a and homologs thereof is measured by Q-PCR.

36. The method of claim 33, wherein said expression level of said one or more gene products selected from the group consisting of HM74, HM74a and homologs thereof is measured by immunohistochemistry.

37. The method of claim 33, wherein said one or more gene products is HM74.

38. The method of claim 33, wherein said one or more gene products is HM74a.

39. A method of evaluating whether a test compound is effective at reducing the symptoms of an inflammatory disorder, said method comprising:
determining whether said test compound binds to one or more gene products selected from the group consisting of HM74, HM74a and homologs thereof; and
evaluating a test compound which binds to one or more gene products selected from the group consisting of HM74, HM74a and homologs thereof in an animal model of said inflammatory disorder.

40. The method of claim 39 further comprising determining whether said test compound reduces the expression level of said one or more gene products selected from the group consisting of HM74, HM74a and homologs thereof in cuboidal endothelial cells in said animal model.

41. The method of claim 39, wherein said animal model is a mouse model.

42. The method of claim 39, wherein said inflammatory disorder is rheumatoid arthritis.

43. The method of claim 39, wherein said test compound is a small molecule.

44. The method of claim 39, wherein said test compound is a natural product.

45. The method of claim 39, wherein said test compound is a macromolecule.

46. The method of claim 45, wherien said macromolecule is a polypeptide.

47. The method of claim 46, wherein said polypeptide is an antibody.


SEQUENCE LISTING (23 sequences)

SEQ ID NO: 1 - DNA, 2051 bp, Homo sapiens
  HM74
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatgcgatttagggaaactgtggcagatgagtgggagactggttgcaaggtgtgaccgcaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactgggaggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaagctttagcagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttgcttttaccattaaaagggaaacgtgcctctgccccacgggtagaggggtgcacgttcctcctggttccttcgcttgtgtttctgtacttaccaaaaatctaccacttcaataaattttgataggagacaaaaaaaaaa

SEQ ID NO: 2 - DNA, 1164 bp, Homo sapiens
  HM74
atgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaa

SEQ ID NO: 3 - protein, 387 aa, Homo sapiens
  HM74
MNRHHLQDHFLEIDKKNCCVFRDDFIAKVLPPVLGLEFIFGLLGNGLALWIFCFHLKSWKSSRIFLFNLAVADFLLIICLPFVMDYYVRRSDWNFGDIPCRLVLFMFAMNRQGSIIFLTVVAVDRYFRVVHPHHALNKISNWTAAIISCLLWGITVGLTVHLLKKKLLIQNGPANVCISFSICHTFRWHEAMFLLEFLLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINRCLQRKMTGEPDNNRSTSVELTGDPNKTRGAPEALMANSGEPWSPSYLGPTSNNHSKKGHCHQEPASLEKQLGCCIE

SEQ ID NO: 4 - DNA, 2082 bp, Homo sapiens
  HM74A
accacacagacacacacctccttgctggagcattcactaggcgaggcgctccatcggactcactagccgcactcatgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaaataaccatgccaagaagggacattgtcaccaagaaccaggatctctggagaaacagttgggctgttgcatcgagtaatgtcactggactcggcctaaggtttcctggaacttccagattcagagaatgcgatttagggaaacggtggcagatgagtgggagactggttgcaaggtgtgaccgcaggaatcctggaggaatagagagtaaagcttctaggcatctgaaacttttgcttcatctctgacgctcgcaggactgaagatgggcaaattgtaggcatttctgctgagcagagttggagccagagatctacttgtgacttgttggccttcttcccacatctgcctcagactggagggggctcagctcctggggtgatatctagcctgcttgtgagctctagcagggataaggagagctgagattggagggaattgtgttgctcctggagggagcccaggcatcattaaacaagccagtaggtcacctggcttccgtggaccaattcatctttcagacaagctttagcagaaatggactcagggaagagactcacacgctttggttaatatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgggcagttaaaacggcttcaggatagaaagctgtttcccacctctgtttgcttttaacattaaaagggaaatgtgcctctgccccacagttagaggggtgcacgttcctcctggttccttcgcttgtgtttctgtacttaccaaaaatctaccatttcaataaattttgataggagacaaaaaaaaaaaaaaaaaa

SEQ ID NO: 5 - DNA, 1092 bp, Homo sapiens
  HM74A
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 6 - protein, 363 aa, Homo sapiens [sequence omitted]

SEQ ID NO: 7 - DNA, 2051 bp, Homo sapiens
  HM74 SNP variant
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatctgatttagggaaactgtggcagatgagtgggagactggttgcaaggtgtgaccgcaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactgggaggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaagctttagcagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttgcttttaccattaaaagggaaacgtgcctctgccccacgggtagaggggtgcacgttcctcctggttccttcgcttgtgtttctgtacttaccaaaaatctaccacttcaataaattttgataggagacaaaaaaaaaa

SEQ ID NO: 8 - DNA, 2051 bp, Homo sapiens
  HM74 SNP variant
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatgcgatttagggaaactgtggcagatgagtgggagactggttgcaaggtgtgaccgcaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactggggggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaagctttagcagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttgcttttaccattaaaagggaaacgtgcctctgccccacgggtagaggggtgcacgttcctcctggttccttcgcttgtgtttctgtacttaccaaaaatctaccacttcaataaattttgataggagacaaaaaaaaaa

SEQ ID NO: 9 - DNA, 2051 bp, Homo sapiens
  SNP variant
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatgcgatttagggaaactgtggcagatgagtgggagactggttgcaaggtgtgaccgcaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactgggaggggctcagctcctcgggtgatatctagcctgcttgtgagctctagcagggataaggagagctgagattggagggaattgtgttgctcctggagggagcccaggcatcattaaacaagccagtaggtcacctggcttccgtggaccaattcatctttcagacaagctttagcagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttgcttttaccattaaaagggaaacgtgcctctgccccacgggtagaggggtgcacgttcctcctggttccttcgcttgtgtttctgtacttaccaaaaatctaccacttcaataaattttgataggagacaaaaaaaaaa

SEQ ID NO: 10 - DNA, 2051 bp, Homo sapiens
  HM74 SNP variant
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatgcgatttagggaaactgtggcagatgagtgggagactggttgcaaggtgtgaccgcaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactgggaggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaatctttagcagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttgcttttaccattaaaagggaaacgtgcctctgccccacgggtagaggggtgcacgttcctcctggttccttcgcttgtgtttctgtacttaccaaaaatctaccacttcaataaattttgataggagacaaaaaaaaaa

SEQ ID NO: 11 - DNA, 2051 bp, Homo sapiens
  HM74 SNP variant
cgccactttgctggagcattcactaggcgaggcgctccatcggactcactagccgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatgcgatttagggaaactgtggcagatgagtgggagactggttgcaaggtgtgaccgcaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactgggaggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaagctttagcagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttgcttttaccattaaaagggaaatgtgcctctgccccacgggtagaggggtgcacgttcctcctggttccttcgcttgtgtttctgtacttaccaaaaatctaccacttcaataaattttgataggagacaaaaaaaaaa

SEQ ID NO: 12 - DNA, 2068 bp, Homo sapiens
  HM74 variant - BC047891
cgccactttgctggagcattcactaggcgaggcgctccatcggactcactagctgcactcatgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaagtttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaatgtcactggactcggcctaaggtttcctggaacttccagattcagagaatctgatttagggaaactgtggcagatgagtgggagactggttgcaaggtgtgaccgcaggaatcctggaggaacagagagtaaagcttctaggcatctgaaacttgcttcatctctgacgctcgcaggactgaagatgggcaaattgtaggcgtttctgctgagcagagttggagccagagatctacttgtgacttgttggccttcttcccacatctgcctcagactggggggggctcagctcctcgggtgatatctagcctgcttgtgagctctagcagggataaggagagctgagattggagggaattgtgttgctcctggagggagcccaggcatcattaaacaagccagtaggtcacctggcttccgtggaccaattcatctttcagacaatctttagcagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttgcttttaccattaaaagggaaatgtgcctctgccccacagttagaggggtgcacgttcctcctggttccttcgcttgtgtttctgtacttaccaaaaatctaccacttcaataaattttgataggagacaaaaaaaaaaaaaaaaaaaaaaaaaaa

SEQ ID NO: 13 - DNA, 1164 bp, Homo sapiens
  HM74 variant - BC047891 - CDS
atgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaagtttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaa

SEQ ID NO: 14 - protein, 387 aa, Homo sapiens
  HM74 variant - BC047891 - translation
MNRHHLQDHFLEIDKKNCCVFRDDFIAKVLPPVLGLEFIFGLLGNGLALWIFCFHLKSWKSSRIFLFNLAVADFLLIICLPFVMDYYVRRSDWKFGDIPCRLVLFMFAMNRQGSIIFLTVVAVDRYFRVVHPHHALNKISNWTAAIISCLLWGITVGLTVHLLKKKLLIQNGPANVCISFSICHTFRWHEAMFLLEFLLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINRCLQRKMTGEPDNNRSTSVELTGDPNKTRGAPEALMANSGEPWSPSYLGPTSNNHSKKGHCHQEPASLEKQLGCCIE

SEQ ID NO: 15 - DNA, 29 bp, Artificial Sequence
  T7 promoter sequence
ggatcctaatacgactcactatagggaga

SEQ ID NO: 16 - DNA, 20 bp, Artificial Sequence
  synthetic PCR primer sequence
gtgtgcatcagcttcagcat

SEQ ID NO: 17 - DNA, 20 bp, Artificial Sequence
  synthetic PCR primer sequence
gaggcagatgtgggaagaag

SEQ ID NO: 18 - DNA, 21 bp, Artificial Sequence
  synthetic PCR primer sequence
gcccaacctcaaataaccatt

SEQ ID NO: 19 - DNA, 20 bp, Artificial Sequence
  synthetic PCR primer sequence
tctctggctccaactctgct

SEQ ID NO: 20 - DNA, 19 bp, Artificial Sequence
  synthetic PCR primer sequence
ctgggcccaacctctcctt

SEQ ID NO: 21 - DNA, 24 bp, Artificial Sequence
  synthetic PCR primer sequence
gcaaaagtttcagatgcctagaag

SEQ ID NO: 22 - DNA, 20 bp, Artificial Sequence
  synthetic PCR primer sequence
gagtcaacggatttggtcgt

SEQ ID NO: 23 - DNA, 20 bp, Artificial Sequence
  synthetic PCR primer sequence
gacaagcttcccgttctcag